(19) United States
(12) Patent Application Publication
(10) Pub. No.: US 2021/0309984 A1
(43) Pub. Date: Oct. 7, 2021

(54) ChiA Enzyme

(71) Applicants: King's College London, London (GB); Northwestern University, Evanston, IL (US)

(72) Inventors: James Garnett, London (GB); Nicholas P. Cianciotto, Chicago, IL (US)

(21) Appl. No.: 17/223,743

(22) Filed: Apr. 6, 2021

Related U.S. Application Data
(60) Provisional application No. 63/005,592, filed on Apr. 6, 2020.

(51) Int. Cl. C12N 9/42; G01N 33/573

(52) U.S. Cl. CPC C12N 9/2442; A61K 38/00; G01N 33/573; C12Y 302/01014

(57) ABSTRACT

There is disclosed a peptide with mucolytic activity, The peptide comprising an amino acid sequence having at least 60% sequence identity to the amino add sequence defined in SEQ ID NO: 1, but does not consist of the amino sequence defined in SEQ ID NO: 2.

### RELATED APPLICATIONS

This application claims the benefit under 35 USC 119(e) of U.S. Provisional Application No. 63/005,592, filed on Apr. 6, 2020, which is incorporated herein by reference in its entirety.

### GOVERNMENT SUPPORT

This invention was made with government support under award number AI043987 awarded by the National Institutes of Health (NIH). The government has certain rights in the invention.

### INCORPORATION BY REFERENCE OF MATERIAL IN ASCII TEXT FILE

This application incorporates by reference the Sequence Listing contained in the following ASCII text file:

File name: 0398-0001US1_SequenceListing_PN836686USA.TXT; created 26 May 2021, 146 KB in size.

### FIELD OF THE INVENTION

The present invention provides peptides having mucolytic activity. The invention also relates to pharmaceutical compositions comprising such peptides and nucleic acid molecules encoding for such peptides. Additionally within the scope of the invention are medical and non-medical uses of said peptide, including treatment of chronic inflammatory lung disease such as asthma, and use of the peptide in in vitro assays.

### BACKGROUND OF THE INVENTION

Chronic inflammatory lung diseases, which include bronchial asthma, chronic obstructive pulmonary disease, bronchiectasis and cystic fibrosis, are a major global health problem and their occurrence have been on the rise for several decades. A key feature of these diseases is the excessive production of airway mucus (1). Asthma for example affects more than 300 million people across the world with symptoms including dyspnoea, coughing and wheezing. The development of asthma is associated with both acute and chronic lung inflammation. This results hi structural changes in the airway and subsequent bronchoconstriction and obstruction of the airway due to mucus hypersecretion (2). In fatal asthma, mucus hypersecretion has been shown to have a major role in death, with overproduction also highly prevalent in mild to moderate asthma (3).

Mucus is an essential component of the lung and functions to moisturise and lubricate the airways. In addition, mucus captures bacteria and other inhaled irritants, which are then removed by the mucociliary transport system. However, overproduction and hypersecretion of mucus results in obstruction of the airway and impairs correct mucociliary function. A major component of mucus and mucosal membranes are heavily glycosylated proteins called mucins, which are either secreted as gel-forming glycoproteins by goblet cells or anchored to the cell surfaces of goblet, mucosal and absorptive epithelial cells. Mucins with variant structures and glycosylation patterns are differentially expressed throughout the body. For example, MUC1, MUC5AC, and MUC6 are the main components of the mucus layer in the stomach, while MUC2 is the most abundant mucin in the small intestine and the colon. In the mucus layer of healthy human airways MUC5B and MUC5AC are the predominant gel-forming mucins but during the progression of asthma and other chronic inflammatory lung diseases, the production of MUC5AC is significantly increased (4, 5). Similarly MUC5AC is increased in other diseases such as rhinosinusitis, Extramammary Paget's disease (EMPD), gallstone disease and pancreatic cancer, in addition to other increased mucin in, for example, inflammatory bowel disease (IBD).

The common treatments for chronic inflammatory lung diseases are bronchodilators and anti-inflammatory agents (e.g. asthma, chronic obstructive pulmonary disease) and broadly active mucolytic agents. However, these approaches are often temporary, incomplete and/or non-specific (6).

One option for treatment is mucolytics. These drugs have the effect of thinning the mucus; although their mechanism of action is vague, they may alter mucin expression. Examples of mucolytics include N-acetylcysteine, Eerdosteine and Ambroxol.

Another option for treatment are inhaled corticosteroids. These are anti-inflammatory drugs and currently the most effective and commonly used long-term control medications for asthma. They reduce swelling and tightening in the airways, although in children, long-term use of inhaled corticosteroids can delay growth slightly. Other side effects can include mouth and throat irritation and oral yeast infections. Examples of corticosteroids are Fluticasone, Budesonide, Mometasone, Beclomethasone, and Ciclesonide. Alternatively, oral corticosteroids can be used to treat serious asthma attacks. Long-term use can cause side effects including cataracts, thinning bones (osteoporosis), muscle weakness, decreased resistance to infection, high blood pressure and reduced growth in children. Examples include Prednisone and Methylprednisolone.

Yet another option for treatment is leukotriene modifiers. These block the effects of leukotrienes and can help prevent symptoms for up to 24 hours. However, in rare cases the drug montelukast is linked to psychological reactions, such as agitation, aggression, hallucinations, depression and suicidal thinking. Examples of these treatments include Montelukast, Zafirlukast, and Zileuton.

Another option is long-acting beta agonists (LABAs). These are bronchodilators that open airways and reduce swelling for at least 12 hours. They are used on a regular schedule to control moderate to severe asthma and to prevent night-time symptoms. They are effective but have been linked to severe asthma attacks. One example of LABAs is Salmeterol. Theophylline is a bronchodilator that is taken daily in pill form to treat mild asthma. Theophylline relaxes the airways and decreases the lungs' response to irritants. It can be helpful for nighttime asthma symptoms. Potential side effects of theophylline include insomnia and gastroesophageal reflux.

Quick-relief medications open the lungs by relaxing airway muscles and can ease worsening symptoms or stop an asthma attack in progress. They start working within minutes but are only effective for four to six hours and are not for daily use. Possible side effects include jitteriness and palpitations. Examples include Albuterol and Levalbuterol.

Accordingly, there is a need to provide further treatments for mucus driven diseases, such as asthma.

A broad range of organisms produce chitinase enzymes that digest chitin, the second most abundant carbohydrate on earth. In a 2006 publication (7), in an entirely different field of research, it has been discovered that the human lung pathogen Legionella pneumophila secretes a unique chitinase enzyme (ChiA) and it has been shown that ChiA promotes bacterial survival in the (murine) lung, and that it is expressed in the infected mouse.

### SUMMARY OF THE INVENTION

The present invention arises from the identification of a functional C-terminal fragment of an enzyme found in Legionella pneumophila which is capable of degrading mucus (i.e. is mucolytic), and specifically is capable of degrading MUC5AC which has an important association with disease, particularly asthma.

Accordingly, in one aspect of the invention there is provided a peptide comprising an amino acid sequence having at least 60% sequence identity to the amino acid sequence defined in SEQ ID NO: 1, wherein the peptide has mucolytic activity, wherein the peptide does not consist of the amino sequence defined in SEQ ID NO: 2.

In some embodiment, the amino acids at positions equivalent to positions 81, 83, 121, 124, 160, 172, and 194 of SEQ ID NO: 1 are identical to or similar to positions 81, 83, 121, 124, 160, 172, and 194 of SEQ ID NO: 1.

The skilled person will also understand that the amino acid at these positions of SEQ ID NO: 1 are also equivalent to positions 504, 506, 544, 547, 583, 595 and 617 of SEQ ID NO: 2 respectively. Therefore, the amino acids at positions equivalent to positions 504, 506, 544, 547, 583, 595 and 617 of SEQ ID NO: 2 are identical to or similar to positions 504, 506, 544, 547, 583, 595 and 617 of SEQ ID NO: 2.

In some embodiments, the peptide consists of an amino acid sequence of no more than 1000 amino acids.

In further embodiments, the peptide retains said mucolytic activity for at least four weeks at 25° C.

In some embodiments, the peptide degrades MUC5AC.

In certain embodiments, the peptide is conjugated to at least one other moiety.

In another aspect of the invention, there is provided a nucleic acid comprising a nucleotide sequence which encodes the peptide as described above.

In a further aspect of the invention, there is provided a nucleic acid comprising a nucleic acid sequence having at least 60% sequence identity to the nucleic acid sequence defined in SEQ ID NO: 3, wherein the resulting peptide has mucolytic activity, wherein the nucleic acid does not consist of the nucleic acid sequence defined in SEQ ID NO: 4.

In some embodiments, the nucleic acid consists of a nucleic acid sequence having at least 70%, 75%, 80%, 85%, 90%, 95% or 99% sequence identity to the nucleic acid sequence defined in SEQ ID NO: 3.

In another aspect of the invention, there is provided a vector comprising a nucleic acid sequence as described above.

In a yet another aspect of the invention, there is provided a host cell comprising a vector described above, preferably wherein the host cell is E. coli.

In a further aspect of the invention, there is provided a use of a peptide comprising an amino acid sequence having at least 60% sequence identity to the amino acid sequence defined in SEQ ID NO: 1, wherein the peptide has mucolytic activity, or a nucleic acid encoding a peptide having at least 60% sequence identity to the amino acid sequence defined in SEQ ID NO: 1, wherein the peptide has mucolytic activity, in an in vitro assay.

Thus, there is also provided a method of performing an in vitro assay, a peptide comprising an amino acid sequence having at least 60% sequence identity to the amino acid sequence defined in SEQ ID NO: 1, wherein the peptide has mucolytic activity, or a nucleic acid encoding a peptide having at least 60% sequence identity to the amino acid sequence defined in SEQ ID NO: 1, wherein the peptide has mucolytic activity. In some embodiments, this method comprises contacting the peptide or the nucleic acid described above with a sample and detecting interaction between the peptide and a mucin in the sample.

In a further aspect of the invention, there is provided a use of a peptide comprising an amino acid sequence having at least 60% sequence identity to the amino acid sequence defined in SEQ ID NO: 1, wherein the peptide has mucolytic activity, or a nucleic acid encoding a peptide having at least 60% sequence identity to the amino acid sequence defined in SEQ ID NO: 1, wherein the peptide has mucolytic activity, in therapy.

In another aspect of the invention, there is provided a peptide comprising an amino acid sequence having at least 60% sequence identity to the amino acid sequence defined in SEQ ID NO: 1 wherein the peptide has mucolytic activity, or a nucleic acid encoding a peptide having at least 60% sequence identity to the amino acid sequence defined in SEQ ID NO: 1, wherein the peptide has mucolytic activity, for use as a medicament.

In yet another aspect of the invention, there is provided a pharmaceutical composition comprising a peptide comprising an amino acid sequence having at least 60% sequence identity to the amino acid sequence defined in SEQ ID NO: 1 wherein the peptide has mucolytic activity, or a nucleic acid encoding a peptide having at least 60% sequence identity to the amino acid sequence defined in SEQ ID NO: 1, wherein the peptide has mucolytic activity, and at least one pharmaceutically acceptable carrier, diluent or excipient.

In some embodiments, the peptide, the use or the pharmaceutical composition described above comprises an amino acid sequence having at least 70%, 75%, 80%, 85%, 90%, 95% or 99% sequence identity to the amino acid sequence defined in SEQ ID NO: 1.

In some embodiments, the peptide, nucleic acid or pharmaceutical composition described above is for use in the treatment of a disease or condition characterised by an increased level of mucin.

In a further aspect of the invention, there is provided a method of treatment of a disease or condition characterised by an increased level of mucin, the method comprising the step of administering:
a peptide comprising an amino acid sequence having at least 60% sequence identity to the amino acid sequence defined in SEQ ID NO: 1 wherein the peptide has mucolytic activity, or
a nucleic acid encoding a peptide having at least 60% sequence identity to the amino acid sequence defined in SEQ ID NO: 1, wherein the peptide has mucolytic activity, or
a pharmaceutical composition comprising a peptide comprising an amino acid sequence having at least 60% sequence identity to the amino acid sequence defined in SEQ ID NO: 1 wherein the peptide has mucolytic activity, or
   a nucleic acid encoding a peptide having at least 60% sequence identity to the amino acid sequence defined in SEQ ID NO: 1, wherein the peptide has mucolytic activity, and at least one pharmaceutically acceptable carrier, diluent or excipient,
      to a patient in need thereof.

In some embodiments, the use, peptide for use, the pharmaceutical composition or the method is as described above, and wherein amino acids at positions equivalent to positions 81, 83, 121, 124, 160, 172, and 194 of SEQ ID NO: 1 are identical to or similar to positions 81, 83, 121, 124, 160, 172, and 194 of SEQ ID NO: 1.

In some embodiments, the disease or condition is further characterised by an increased level of MUC5AC. In further embodiments, the disease or condition is a chronic inflammatory lung disease, rhinosinusitis, extramammary Paget's disease, gallstone disease, pancreatic cancer or inflammatory bowel disease. In preferred embodiments, the disease or condition is asthma.

Definitions

In this specification, the following terms may be understood as follows:

The term “peptide” as used herein, refers to a polymer of amino acid residues that is (or has a sequence that corresponds to) a fragment of a longer protein. The term also applies to amino acid polymers in which one or more amino acid residues is a modified residue, or a non-naturally occurring residue, such as an artificial chemical mimetic of a corresponding naturally-occurring amino acid, as well as to naturally occurring amino acid polymers. The term “fragment”, as used herein, refers to a series of consecutive amino acids from a longer polypeptide or protein. In some embodiments, the peptide or the fragment is isolated, that is to say, the peptide or fragment is removed from the components in its natural environment.

The term “amino acid” as used herein refers to naturally occurring and synthetic amino acids, as well as amino acid analogues and amino acid mimetics that have a function that is similar to naturally occurring amino acids. Naturally occurring amino acids are those encoded by the genetic code, as well as those modified after translation in cells (e.g. hydroxyproline, gamma-carboxyglutamate, and O-phosphoserine). The phrase “amino acid analogue” refers to compounds that have the same basic chemical structure (an alpha carbon bound to a hydrogen, a carboxy group, an amino group, and an R group) as a naturally occurring amino acid but have a modified R group or modified backbones (e.g. homoserine, norleucine, methionine sulfoxide, methionine methyl sulphonium). The phrase “amino acid mimetic” refers to chemical compounds that have different structures from, but similar functions to, naturally occurring amino acids. It is to be appreciated that, owing to the degeneracy of the genetic code, nucleic acid molecules encoding a particular polypeptide may have a range of polynucleotide sequences. For example, the codons GCA, GCC, GCG and GCT all encode the amino acid alanine.

The percentage “identity” between two sequences may be determined using the BLASTP algorithm version 2.2.2 (Altschul, Stephen F., Thomas L. Madden, Alejandro A. Schäffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402) using default parameters. In particular, the BLAST algorithm can be accessed on the internet using the URL http://www.ncbi.nlm.nih.gov/blast/.

The terms “gene”, “polynucleotides”, and “nucleic acid molecules” are used interchangeably herein to refer to a polymer of multiple nucleotides. The nucleic acid molecules may comprise naturally occurring nucleic acids (i.e. DNA or RNA) or may comprise artificial nucleic acids such as peptide nucleic acids, morpholin and locked nucleic acids as well as glycol nucleic acids and threose nucleic acids.

The term “nucleotide” as used herein refers to naturally occurring nucleotides and synthetic nucleotide analogues that are recognised by cellular enzymes.

The term “vector” as used herein refers to any natural or artificial construct containing a nucleic acid molecule in which the nucleic acid molecule can be subject to cellular transcription and/or translation enzymes. Exemplary vectors include: a plasmid, a virus (including bacteriophage), a cosmid, an artificial chromosome or a transposable element.

The term “host cell” as used herein refers to any biological cell which can be cultured in medium and used for the expression of a recombinant gene. Such host cells may be eukaryotic or prokaryotic and may be a microorganism such as a bacterial cell, or may be a cell from a cell line (such as an immortal mammalian cell line).

The term “mucolytic activity” refers to the capability of the peptide of the invention to degrade at least one mucin as defined herein. Mucins are a glycoprotein constituent of mucus known to the skilled person in the art, but include human mucins MUC1, MUC2, MUC5AC, MUC5B, and MUC7, and include any other mucin known to the person skilled in the art. Mucolytic activity may be indiscriminate (i.e. degrade all mucins) or discriminate (i.e. degrade a particular mucin). Mucolytic activity can be measured by any means known to the skilled person, including the assays described herein, e.g. incubating a peptide with mucin extracts, and immunoblotting this to analyse the degraded products, as described in the Materials and Methods section of the Example below.

The term “degrade” or “degrading” refer to the capability of the peptide of the invention to break down a mucin, or to reduce one compound to a less complex compound.

The term “retains” means that the peptide maintains the desired mucolytic activity at least 10%, 20%, 30%, 40%, 50%, 60%, 70%, 80%, 90%, 95% or 99% of a control peptide. In some examples, the peptide may have 100% of the mucolytic activity level as a control peptide. In one example, the control peptide is the peptide at time=0, or the control peptide has been stored in activity-preserving conditions, such as in a −70° C. freezer.

The term “conjugated” means reversibly combined or bound.

The term “in vitro assay” means any experiment that is not carried out in a living organism. The person skilled in the art would be well aware of a variety of in vitro assays, but examples include: protein-based assays such as ELISAs, CBAs, ELISpots, immunoblotting, or nucleic acid based assays such as PCR, Northern or Southern blotting,

The terms “treatment” or “therapy” may be used interchangeably and refer to any partial or complete treatment and includes: inhibiting the disease or symptom, i.e. arresting its development; and relieving the disease or symptom, i.e. causing regression of the disease or symptom.

The term “medicament” means a composition suitable for treatment or therapy as defined above.

The term “pharmaceutical composition”, as used herein, means a pharmaceutical preparation suitable for administration to an intended human or animal subject for therapeutic purposes.

The terms “increased” or “increased level” mean an increased level of a substance compared to a resting state, control state or non-disease state. For example, in the context of a diseased patient such as an asthma patient, there may be an increased level of mucus compared to the level of mucus when the disease is not active, when the disease is being controlled by treatment, or where the disease is not present (e.g. the level in a healthy patient). An increased level may comprise an increase of at least 10%, 20%, 30%, 40%, 50%, 60%, 70%, 80%, 90%, 100%, 200%, 300%, 400% or 500% compared to a resting state, control state or non-disease state as defined above. Alternatively, an increased level comprises an increase of at least 1 fold, 2 fold, 3 fold, 4 fold, 5 fold, 10 fold, 15 fold, or 20 fold compared to a resting state, control state or non-disease state as defined above. Alternatively still, an increased level comprises an increase compared to a resting state, control state or non-disease state as defined above wherein said increase is statistically significant (P<0.05) when analysed by student T-test.

The term “chronic inflammatory lung disease” as used herein refers to any chronic disease or condition affecting the respiratory system that is associated with mucus. Chronic inflammatory lung disease includes asthma, chronic obstructive pulmonary disease, bronchiectasis and cystic fibrosis.

### BRIEF DESCRIPTION OF THE FIGURES

FIGS. 1A-1D show chitin binding and endochitinase functions of ChiA.

FIG. 1A shows a schematic representation of ChiA with domain boundaries annotated. FIG. 1B shows that ChiA-FL, subdomains (NT, N1, N2, N3, CTD) and ChiA-CTD mutants (E543M, Q583A, Q617A) were assayed for chitinase activity against p-NP-[GlcNAc]₃. Data represent the mean and standard deviation for triplicate experiments. FIG. 1C shows a chitin pull-down experiment to assess direct interactions between immobilized chitin and ChiA. ChiA-FL and subdomains (NT, N1, N2, N3, CTD, CTD^{E543M}) were incubated with chitin beads and analysed by SDS-PAGE. BSA was used as a control. L: loaded sample; B: eluted beads. Eluted samples undergo an upward shift compared to the input sample due to differences in buffer conditions. Data is representative of three independent repeat experiments. FIG. 1D shows initial in silico docking studies of zinc (sphere) and T-antigen from MUC5AC (sticks) to ChiA-CTD (surface).

FIGS. 2A-2F show crystal structure of ChiA-CTD.

FIG. 2A shows a cartoon representation of ChiA-CTD with secondary structure and extended loops annotated. Additional ChiA-CTD α3-helix is highlighted with an asterisk. FIG. 2B shows the same representation rotated by 180°. FIG. 2C shows a stick representation of the ChiA-CTD active site superimposed with ChiNCTU2 E145GN227F mutant in complex with chitotetraose (PDB ID code 3n18). ChiA-CTD and ChiNCTU2 are drawn as sticks with chitotetraose shown as ball and stick. Mutated residues in ChiNCTU2 are indicated and carbohydrate positions relative to the hydrolysed glycosidic bond are numbered. FIG. 2D shows the same representation as FIG. 2C, without the mutated residue annotations. FIG. 2E shows a model of ChiA-CTD as a cartoon and FIG. 2F shows electrostatic surface potential, bound to chitotetraose drawn as spheres.

FIGS. 3A-3D show model of ChiA-FL in solution.

FIG. 3A shows an initial model of ChiA. Linkers are shown as individual grey spheres. FIG. 3B shows a ChiA enemble model (line) fit to the ChiA SAXS data (black open circles) with χ² of 1.09. FIG. 3C shows three independent ensemble optimization method runs (pale grey, medium grey and dark grey) yielded similar distributions of three populations. FIG. 3D shows sample ChiA models corresponding to the centre of each population for all three runs.

FIG. 4 shows mucin binding of ChiA. ELISA analysis of binding between immobilised type I-S, II or III porcine stomach mucins and His-tagged ChiA-FL, subdomains (NT, N1, N2, N3, CTD) and positive (SsIE) and negative (NttE) controls, detected with anti-His-tag antibody. SsIE was used as a positive control as this is an enzyme with known mucolytic activity. BSA-coated wells were also used as controls. ***P<0.001; verses control empty well by two-tailed Student's test.

FIGS. 5A-5D show mucinase activity of ChiA.

FIG. 5A shows secreted mucinase activity of L. pneumophila wild-type and chiA mutant strains in an immunoblot of type II porcine stomach mucins (200 μg) incubated with either BYE medium alone (BYE), a cocktail of known mucinase enzymes added to BYE medium (cocktail), or supernatants from BYE cultures of wild-type 130b (WT) or chiA mutant NU318 (ΔchiA). Asterisk highlights a lower-MW (˜200 kDa) mucin species generated by the cocktail that is not present in the supernatant samples. FIG. B shows an immunoblot of type II porcine stomach mucins (400 μg) incubated with either supernatants from BYE cultures of wild-type 130b (WT) or chiA mutant NU318 (AchiA). White arrow highlights ChiA-dependent mucin fragment (˜95 kDa) and black arrows highlight non-ChiA dependent mucin fragments (˜100 and ˜90 kDa). The data presented are representative of three independent experiments. FIG. 5C shows a mucin penetration assay of L. pneumophila wild-type (WT) and chiA mutant (ΔchiA) strains applied to the upper chamber of 3.0 μm transwell coated with type II mucin extract. Bacteria that penetrated the transwell were collected from the lower chamber and plated for CFU. Penetration ratio represents CFU in lower chamber 50 or 100 μg mucin/CFU in lower chamber 0 μg mucin. N=3 experimental replicates. Statistical analysis was done using Two-way ANOVA with Boneferri post-hoc test. Error bars represent standard deviation. *P=<0.05, **P=<0.01. FIG. 5D shows an immunoblot of type II porcine stomach mucin extract incubated with ChiA-FL, subdomains (NTD, CTD), SsIE or buffer alone, +/−EDTA, detected with MUC5AC antibody. The bands at ˜70 kDa and ˜60 kDa correspond to ChiA and SsIE processed MUC5AC fragments, respectively.

FIG. 6 shows ChiA-CTD Zn²⁺ binding sites.

Surface representation of ChiA-CTD showing the spatial distribution of Zn²⁺ ions during MD simulations. The the spatial distribution function (sdf) is represented with isosurfaces connecting points with sdf=20 (mesh) and 30 (dark surface) x average sdf. Zn²⁺ high-density sites (dark spots) around the chitinase and peptidase active sites are numbered 1 to 8. Blow out boxes show representative structures from the MD simulations to illustrate Zn²⁺ binding in the eight regions, with Zn²⁺ ions shown as spheres, their coordinating residues as sticks and ChiA-CTD as cartoon.

FIGS. 7A-7N-show ITC analysis of the interaction of Zn²⁺ with ChiA-CTD. Isothermal titration calorimetry (ITC) was used to measure the affinities of Zn²⁺ for wild-type ChiA-CTD (WT) and ChiA-CTD variants (E543M, E543M/D504A, E543M/H506A, E543M/H544A, E543M/N547A and E543M/Q583A). Raw data (top) and normalized binding curves (bottom) are reported. Black squares indicate the normalized heat of interaction obtained per injection, while a black curve represents the best fit obtained by non-linear least-squares procedures based on a 1:1 binding model. FIG. 7A shows raw data for wild-type ChiA-CTD (WT) and FIG. 7B shows the normalized binding curve for WT. FIG. 7C shows raw data for the E543M mutant and FIG. 7D shows the normalized binding curve for the E543M mutant. FIG. 7E shows raw data for the E543M/D504A mutant and FIG. 7F shows the normalized binding curve for the E543/D504M mutant. FIG. 7G shows raw data for the E543M/H506A mutant and FIG. 7H shows the normalized binding curve for the E543M/H506A mutant. FIG. 71 shows raw data for the E543M/H544A mutant and FIG. 7J shows the normalized binding curve for the E543/H44A mutant. FIG. 7K shows raw data for the E543M/N547A mutant and FIG. 7L shows the normalized binding curve for the E543M/N547A mutant. FIG. 7M shows the raw data for the E543M/Q595A mutant and FIG. 7N shows the normalized binding curve for the E543M/Q595A mutant.

FIGS. 8A-8C show peptidase active site of ChiA.

FIG. 8A shows an immunoblot of type II porcine stomach mucin extract incubated with ChiA-CTD, ChiA-CTD mutants (D504A, H506A, E543M, H455A, N547A, Q583A, Q595A, Q617A) or buffer alone and detected with MUC5AC antibody. The band at ˜75 kDa corresponds to a ChiA processed MUC5AC fragment. FIG. 8B shows a surface and cartoon representation of ChiA-CTD bound to chitotetraose (sticks) highlights the expected mucin recognition site. Residues that bind Zn²⁺, form the metal-dependent aminopeptidase active site and form the chitinase active site are annotated. FIG. 8C shows an expanded view of the active site of the ChiA structure shown in FIG. 8B.

FIG. 9 shows mucin binding of ChiA-CTD mutants.

ELISA analysis of binding between immobilised type II or III mucin extracts and His-tagged wild-type ChiA-CTD (WT), ChiA-CTD mutants (D504A, H506A, E543M, H544A, N547A, Q583A, Q595A, Q617A) and controls (SsIE, NttE). Anti-His-tag antibody conjugated to HRP was used to measure OD450 nm values. BSA-coated wells were used as controls. Data represent the mean and standard deviation for triplicate experiments. *, P<0.001; verses control empty well by two-tailed Student's test.

### DETAILED DESCRIPTION OF THE INVENTION

Using chitin binding and chitinase activity assays, it is shown that ChiA-N1 is a chitin binding module and confirmed that ChiA-CTD (C-terminal chitinase domain) is a glycosyl hydrolase domain. Using binding assays, it is shown that ChiA-N1, ChiA-N2, ChiA-N3 domains and ChiA-CTD can associate with mucin glycoproteins. It is further shown through structural and biochemical studies that ChiA-CTD has novel peptidase activity against mucin glycoproteins, which is independent of its chitinase active site.

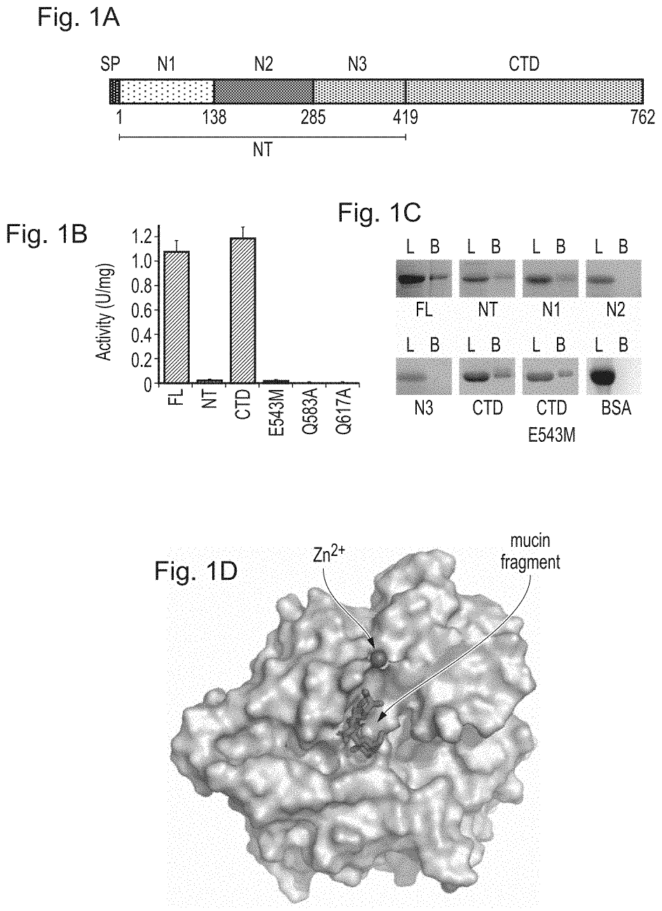

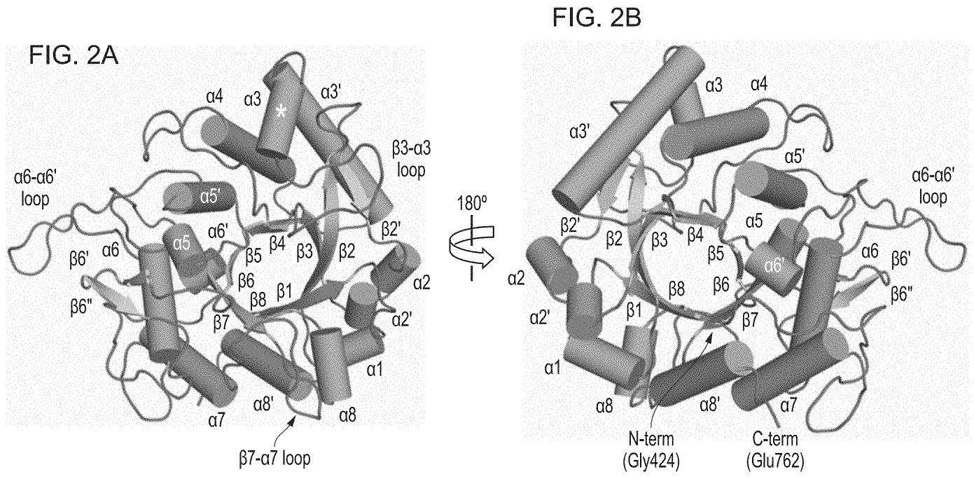

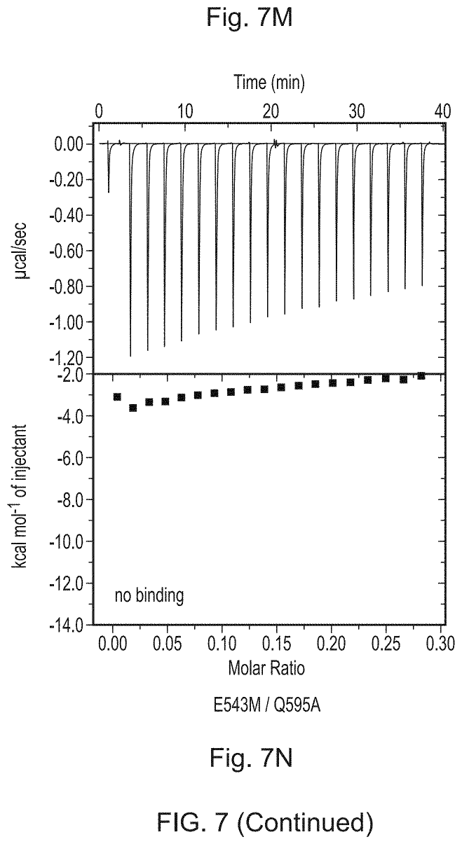

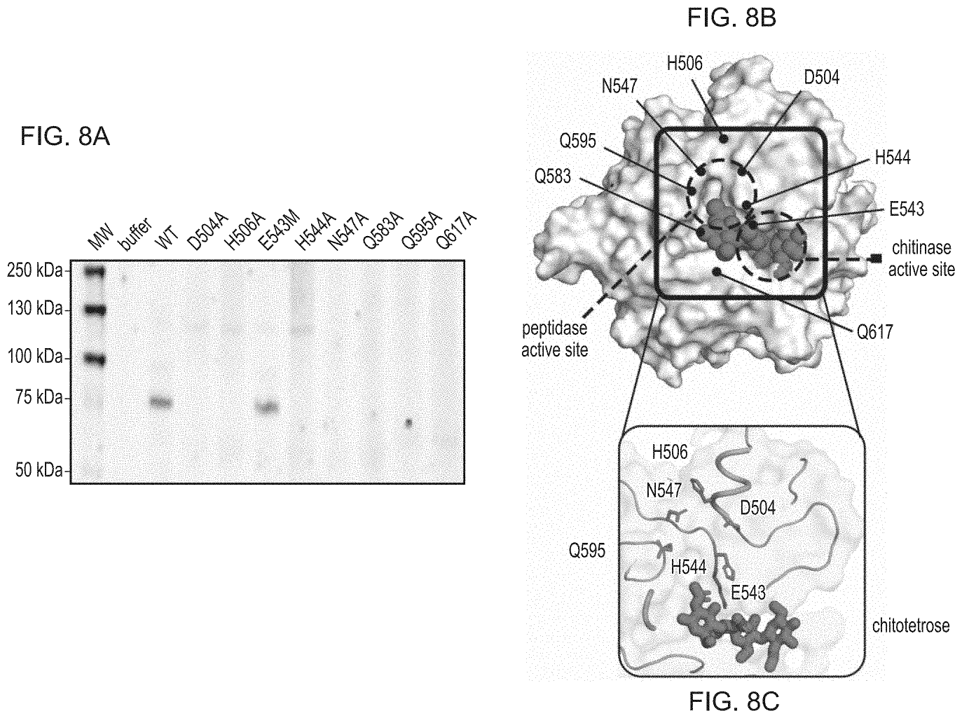

A novel molecular mechanism has been elucidated where the C-terminal chitinase domain of L. pneumophila ChiA (ChiA-CTD) has additional unique Zn²⁺-dependent peptidase activity against MUC5AC (FIG. 8A, 8B, 8C, 9). Here, the peptidoglycan backbone is recognized by ChiA-CTD, primarily through carbohydrate interactions, and the peptide backbone of the mucin is then cleaved (FIG. 1D). Although this enzyme mechanism is similar to the M60-family of peptidases (12), the ChiA-CTD fold is not shared with this family. Whilst ChiA (SEQ ID NO: 2) and ChiA-CTD (SEQ ID NO: 40) show mucolytic activity, ChiA-N1, ChiA-N2 and ChiA-N3 do not show mucolytic activity (SEQ ID NOs: 7, 9, and 11 respectively).

ChiA-CTD therefore has an application in disease where there is an increased level of mucus, such as asthma, in order to break down the excess and problematic mucus.

Unlike other M60-family or mucolytic enzymes, ChiA-CTD (SEQ ID NO: 40) is stable and active for at least four weeks at 25° C. and at least several months at −20° C. (>90% activity against MUC5AC). In contrast, full length ChiA (SEQ ID NO: 2) shows signs of breaking down into degraded individual domains after only a few days. Furthermore, with the described bacterial expression system, it is possible to produce high yields of recombinant ChiA-CTD (>20 mg per 1 L culture), which can be isolated in a single step affinity purification step.

As such, the present invention relates to a peptide comprising an amino acid sequence having at least 60% sequence identity to the amino acid sequence defined in SEQ ID NO: 1, wherein the peptide has mucolytic activity, wherein the peptide does not consist of the amino sequence defined in SEQ ID NO: 2. Preferably, the peptide comprises an amino acid sequence having at least 70%, 75%, 80%, 85%, 90%, 95% or 99% sequence identity to the amino acid sequence defined in SEQ ID NO: 1.

Accordingly, in some embodiments, the peptide is not 100% identical to SEQ ID NO: 1, however the person skilled in the art will recognise that peptides having at least 60%, 70%, 75%, 80%, 85%, 90%, 95% or 99% sequence identity to SEQ ID NO: 1, will have the desired function of the invention, or in other words, will have mucolytic activity.

In alternative embodiments, the peptide comprises an amino acid sequence having at least 60%, 70%, 75%, 80%, 85%, 90%, 95% or 99% identity to SEQ ID NO: 40.

In some embodiments, the peptide consists of an amino acid sequence having at least 60%, 70%, 75%, 80%, 85%, 90%, 95% or 99% identity to SEQ ID NO: 1. In alternative embodiments, the peptide consists of an amino acid sequence having at least 60%, 70%, 75%, 80%, 85%, 90%, 95% or 99% identity to SEQ ID NO: 40. Thus in some embodiments, one or more amino acids of the peptides are omitted or are substituted for a different amino acid, preferably a similar amino acid. A similar amino acid is one which has a side chain moiety with related properties and the naturally occurring amino acids may be categorized into the following groups. The group having basic side chains: lysine, arginine, histidine. The group having acidic side chains: aspartic acid and glutamic acid. The group having uncharged polar side chains: aspargine, glutamine, serine, threonine and tyrosine. The group having non-polar side chains: glycine, alanine, valine, leucine, isoleucine, proline, phenylalanine, methionine, tryptophan and cysteine.

Therefore, it is preferred to substitute amino acids within these groups and the substitution of a “similar” amino acid residue is a substitution within one of the aforementioned groups (this is also known as a “conservative substitution”).

In some embodiments, the amino acids at positions equivalent to positions 81, 83, 121, 124, 160, 172, and 194 of SEQ ID NO: 1 (or positions 504, 506, 544, 547, 583, 595 and 617 of SEQ ID NO: 2) are substituted for similar amino acids to those amino acids at positions 81, 83, 121, 124, 160, 172, and 194 of SEQ ID NO: 1 (or positions 504, 506, 544, 547, 583, 595 and 617 of SEQ ID NO: 2).

In some embodiments, the amino acids at positions equivalent to positions 81, 83, 121, 124, 160, 172, and 194 of SEQ ID NO: 1 (or positions 504, 506, 544, 547, 583, 595 and 617 of SEQ ID NO: 2) are identical to positions 81, 83, 121, 124, 160, 172, and 194 of SEQ ID NO: 1 (or positions 504, 506, 544, 547, 583, 595 and 617 of SEQ ID NO: 2). Or in other words, the amino acids at these positions are identical to the equivalent position in the wild type protein (SEQ ID NO: 2).

In some embodiments, the peptide further comprises an insertion, substitution or a deletion of 1, 2, 3, 4 or 5 amino acids in the sequence defined by SEQ ID NO: 1. In further embodiments, said insertion, substitution or deletion is not at positions equivalent to 81, 83, 121, 124, 160, 172, and 194 of SEQ ID NO: 1.

The term “the position equivalent to position 81 . . . of SEQ ID NO: 1”, as used herein, means an amino acid in the peptide of SEQ ID NO:1 located in the peptide's amino acid chain at a position corresponding to the 81^{st} amino acid of the amino acid sequence of SEQ ID NO:1, counting from the N-terminal. Corresponding meanings are attributed to the amino acid equivalent to position 83, 121, 124, 160, 172 and 194. Accordingly, the term “the position equivalent to position 504 . . . of SEQ ID NO: 2”, as used herein, means an amino acid in a peptide of SEQ ID NO: 2 located in the peptide's amino acid chain at a position corresponding to the 504^{th} amino acid of the amino acid sequence of SEQ ID NO: 2, counting from the N-terminal. Again, corresponding meanings are attributed to the amino acid equivalent to 506, 544, 547, 583, 595 and 617 of SEQ ID NO: 2.

In preferred embodiments, the term “the position equivalent to position . . . ” means that the positions immediately adjacent to a position are also identical or similar to the equivalent position in SEQ ID NO: 1 or 2. For example, if position 81 is identical or similar to the equivalent position in SEQ ID NO: 1, then positions 80 and 82 are also identical or similar to the equivalent position in SEQ ID NO: 1.

It is generally preferred that the polypeptide conforms with the chemistry of naturally occurring polypeptides (although it may be synthesized in vitro) but in some alternative embodiments the polypeptide is a peptidomimetic, that is to say a modification of a polypeptide in a manner that will not naturally occur. Such peptidomimetics include the replacement of naturally occurring amino acids with synthetic amino acids and/or a modification of the polypeptide backbone. For example in some embodiments, the peptide bonds are replaced with a reverse peptide bond to generate a retro-inverso peptidomimetic (see Méziére et al J Immunol. 1997 Oct. 1; 159(7):3230-7, which is incorporated herein by reference.) Alternatively, the amino acids are linked by a covalent bond other than a peptide bond but which maintains the spacing and orientation of the amino acid residues forming the polymer chain. In some embodiments, the peptide is bound to at least one zinc ion (Zn²⁺). In some embodiments, the peptides binds to at least one zinc ion (Zn²⁺) when in use. In all embodiments, the peptide will have a level of mucolytic activity as defined herein, or as described above, degrade at least one mucin. Mucolytic activity may be indiscriminate (i.e. degrade all mucins) or discriminate (i.e. degrade a particular mucin).

Mucins are high molecular weight glycoproteins that contain large numbers of heavily O-glycosylated serine/threonine rich repeat sequences. They exist as cell surface exposed transmembrane proteins or secreted gel-forming proteins of the mucosal barrier and act as the first line of defence against bacterial infection. However, overproduction of mucins can be problematic in disease, for example in chronic obstructive pulmonary disease (COPD) or asthma.

In further embodiments, the peptide consists of an amino acid sequence of no more than 400, 500, 600, 700, 800, 900 or 1000 amino acids. Peptides within the scope of the invention maintain the underlying function of the invention, i.e. mucolytic activity, but may have additional amino acids added to either side of SEQ ID NO: 1.

In some embodiments, the peptide retains said mucolytic activity for at least four weeks at 25° C. Stability at this temperature, which may be the temperature of a lab bench, is an advantageous feature to improve application of this peptide to the uses and methods described herein. In other words, the peptide may retain the mucolytic activity when it is left on a lab bench, or a storage shelf or the like, for a number of days or weeks.

Preferably the peptide degrades MUC5AC (SEQ ID NO: 39). MUC5AC is a major mucin expressed in the mammalian airway and lung and has been linked to mucus hypersecretion in the respiratory tract.

In some embodiments, the peptide is conjugated to at least one other moiety. For example, the peptide may be conjugated to at least one PEG or at least one glycan.

In yet another aspect of the invention, there is a nucleic acid comprising a nucleotide sequence which encodes the described peptide. Alternatively, there is a nucleic acid comprising an nucleic acid sequence having at least 60% sequence identity to the nucleic acid sequence defined in SEQ ID NO: 3, wherein the resulting peptide has mucolytic activity, and wherein the nucleic acid does not consist of the nucleic acid sequence defined in SEQ ID NO: 4. Preferably, the nucleic acid, consists of a nucleic acid sequence having at least 70%, 75%, 80%, 85%, 90%, 95% or 99% sequence identity to the nucleic acid sequence defined in SEQ ID NO: 3.

Similarly to above, the nucleic acid sequence may differ from the specific sequences disclosed herein, as such sequences may comprise an nucleic acid sequence having at least 60%, 70%, 75%, 80%, 85%, 90%, 95% or 99% sequence identity to SEQ ID NO: 3, which the person skilled in the art will understand can produce a peptide having the desired function of the invention, or in other words, a peptide having mucolytic activity as defined herein.

In addition to the sequence specifically encoding the protein of the invention, the nucleic acid molecule may contain other sequences such as primer sites, transcription factor binding sites, vector insertion sites and sequences which resist nucleolytic degradation (e.g. polyadenosine tails). The nucleic acid molecule may be DNA or RNA and may include synthetic nucleotides, provided that the polynucleotide is still capable of being translated in order to synthesize a protein of the invention. In another aspect, there is a vector comprising such a nucleic acid sequence; in a further aspect, there is a host cell comprising said vector, preferably wherein the host cell is E. coli.

In addition to the nucleic acid sequence described above, the plasmid comprises other elements such as a prokaryotic origin of replication (for example, the E. coli OR1 origin of replication) an autonomous replication sequence, a centromere sequence; a promoter sequence, upstream of the nucleic acid sequence, a terminator sequence located downstream of the nucleic acid sequence, an antibiotic resistance gene and/or a secretion signal sequence. A vector comprising an autonomous replication sequence is also a yeast artificial chromosome. In some alternative embodiments, the vector is a virus, such as a bacteriophage and comprises, in addition to the nucleic acid sequence of the invention, nucleic acid sequences for replication of the bacteriophage, such as structural proteins, promoters, transcription activators and the like.

The nucleic acid molecule of the invention may be used to transfect or transform host cells in order to synthesize the protein of the invention. Suitable host cells include prokaryotic cells such as E. coli and eukaryotic cells such as yeast cells, or mammalian or plant cell lines. Host cells are transfected or transformed using techniques known in the art such as electroporation; calcium phosphate base methods; a biolistic technique or by use of a viral vector.

After transfection, the nucleic acid molecule of the invention is transcribed as necessary and translated. In some embodiments, the synthesized protein is allowed to remain in the host cell and cultures of the recombinant host cell are subsequently used. In other embodiments, the synthesized protein is extracted from the host cell, either by virtue of its being secreted from the cell due to, for example, the presence of secretion signal in the vector, or by lysis of the host cell and purification of the protein therefrom.

In one aspect of the invention, there is a use of such a peptide, or a nucleic acid as defined herein, in an in vitro assay, alternatively, there is a use of such a peptide or nucleic acid in therapy. The person skilled in the art would be well aware of a variety of in vitro assays, but examples include: protein-based assays such as ELISAs, CBAs, ELISpots, immunoblotting, or nucleic acid based assays such as PCR, northern or southern blotting.

In a further aspect of the invention, there is a peptide or a nucleic acid as described above for use as a medicament. Such a medicament may be administered to a patient in need thereof in any way known to the person skilled in the art. In some embodiments, the medicament may be administered orally or nasally through an inhaler. In other embodiments, the medicament may be administered as a topical solution or spray.

A pharmaceutical composition may comprise a peptide or a nucleic acid of the invention, as well as at least one pharmaceutically acceptable carrier, diluent or excipient. In some embodiments, the at least one pharmaceutically acceptable carrier, diluent or excipient is physiological saline, phosphate buffered saline (PBS) and/or sterile water. In some embodiments, the pharmaceutical composition consists essentially of the peptide of the invention.

In another aspect of the invention, there is a peptide or a pharmaceutical composition as described above, for the treatment of a disease or condition characterised by increased level of mucin. Optionally, the disease or condition is further characterised by an increased level of MUC5AC. For example, the disease or condition may be asthma, which is associated with an increased level of MUC5AC (4). There may be an increased level due to increased production at the mRNA or the protein level and/or due to a reduction in natural degradation, recycling or inhibition of the mRNA or the protein. The level of mucin, or MUC5AC, may increase due to inflammation and/or an allergic response. In one example, there may be an increased level of mucin because of a decreased level of a natural inhibitor of mucin. In another example, there may be upregulation of the mRNA transcript for MUC5AC due to downregulation in a transcriptional regulator of MUC5AC. The person skilled in the art would be well aware of many scenarios in which there may be an increased level of an analyte, including mucin or specifically MUC5AC.

In one embodiment, the disease or condition is a chronic inflammatory lung disease, preferably asthma, chronic obstructive pulmonary disease, bronchiectasis and cystic fibrosis. Asthma is a respiratory condition marked by attacks of spasm in the bronchi of the lungs, causing difficulty in breathing. Mucus can play a role in asthma pathology by plugging and/or obstructing the airways. Chronic obstructive pulmonary disease (COPD), is a chronic inflammatory lung disease that causes obstructed airflow from the lungs. Bronchiectasis is a chronic lung condition where the airways of the lung become abnormally widened, leading to a build-up of excess mucus that can make the lungs vulnerable to infection. Cystic fibrosis is a hereditary disorder affecting the exocrine glands causing production of abnormally thick mucus which can block pancreatic ducts, intestines and bronchi, and often results in respiratory infection.

In another embodiment, the disease or condition is not a chronic inflammatory lung disease. For example, the disease or condition may be rhinosinusitis, extramammary Paget's disease (EMPD), gallstone disease, pancreatic cancer, or inflammatory bowel disease (IBD). The disease or condition may be any other disease or condition known to the skilled person that is associated with increased levels of mucus, and particularly increased levels of MUC5AC.

In one aspect, there is a method of treatment of a disease or condition characterised by an increased level of mucin, the method comprising the step of administering a peptide, pharmaceutical composition, or nucleic acid as described above.

In a preferred embodiment, the disease or condition is characterised by an increased level of MUC5AC as described above. In one embodiment, the disease or condition is a chronic inflammatory lung disease, preferably asthma, chronic obstructive pulmonary disease, bronchiectasis and cystic fibrosis.

In one embodiment, the peptide is delivered at a dose of 0.01 μg-100 μg per kg per day. The skilled person will understand that this represents a suitable dose to obtain a technical effect in an individual to be treated. For example, in the context of asthma, the suitable dose will provide a level of mucolytic activity which is beneficial for a patient suffering from an increased level of mucus, or specifically MUC5AC.

### EXAMPLE

Material and Methods

Cloning, Expression and Purification

Full-length ChiA minus the N-terminal periplasmic signal sequence ChiA (SEQ ID NO: 4; resulting in peptide of SEQ ID NO: 2), the ChiA N-terminal region (SEQ ID NO: 6; resulting in peptide of SEQ ID NO: 5), the ChiA N1-domain (SEQ ID NO: 8; resulting in peptide of SEQ ID NO: 7), the ChiA N2-domain (SEQ ID NO: 10; resulting in peptide of SEQ ID NO: 9), the ChiA N3-domain (SEQ ID NO: 12; resulting in peptide of SEQ ID NO: 11) the ChiA C-domain (SEQ ID NO: 41; resulting in peptide of SEQ ID NO: 40) and NttE (residues 1-269) were amplified from the genomic DNA of L. pneumophila strain 130b and cloned into the N-terminal His6-tagged vector pET-46 Ek/LIC. SsIE (residues 90-1520), minus the N-terminal periplasmic signal sequence and mature SsIE N-terminal proline-rich region, were amplified from the genomic DNA of E. coli strain W and cloned into the C-terminal His6-tagged vector pOPINE. These were transformed into E. coli SHuffle cells (New England Biolabs) and grown at 37° C. in LB media with 100 μg/ml ampicillin. Expression was induced with 1 mM isopropyl-d-1-thiogalactopyranoside (IPTG) at an OD600nm of 0.6 and cells were harvested after growth overnight at 18° C. Cells were resuspended in 20 mM Tris-HCl pH 8, 200 mM NaCl, 5 mM MgCl2, 1 mg/ml DNase I, 5 mg/ml lysozyme, lysed by sonication and purified using nickel affinity chromatography. All samples were then gel filtered using a Superdex 200 column (GE Healthcare) equilibrated in 20 mM Tris-HCl pH 8, 200 mM NaCl.

Site-Directed Mutagenesis

E543M chiA-CTD mutant (SEQ ID NO: 18; resulting in peptide of SEQ ID NO: 17) was created using pET46chiA-CTD template DNA with a QuikChange II Site-Directed Mutagenesis Kit (Stratagene). D504A (SEQ ID NO: 14, resulting in peptide of SEQ ID NO: 13), H506A (SEQ ID NO: 16, resulting in peptide of SEQ ID NO: 15), H544A (SEQ ID NO: 20, resulting in peptide SEQ ID NO: 19), N547A (SEQ ID NO: 22, resulting in peptide of SEQ ID NO: 21), Q583A (SEQ ID NO: 26, resulting in peptide of SEQ ID NO: 25), Q595A (SEQ ID NO: 24, resulting in peptide of SEQ ID NO: 23), Q617A (SEQ ID NO: 28, resulting in peptide of SEQ ID NO: 27), E543M/D504A (SEQ ID NO: 30, resulting in peptide of SEQ ID NO: 29), E543M/H506A (SEQ ID NO: 32, resulting in peptide of SEQ ID NO: 31), E543M/H544A (SEQ ID NO: 34, resulting in peptide of SEQ ID NO: 33), E543M/N547A (SEQ ID NO: 36, resulting in peptide of SEQ ID NO: 35) and E543M/Q595A (SEQ ID NO: 38, resulting in peptide of SEQ ID NO: 37) chiA-CTD mutants were synthesized by Synbio Technologies and cloned into pET28b vector using NcoI and XhoI restriction sites. All resulting clones were verified by DNA sequencing and then expressed and purified as described for wild-type ChiA-CTD (SEQ ID NO: 40).

Chitinase Activity Assay

Enzyme activity was determined using 4-Nitrophenol β-D-N,N′,N″-triacetylchitotriose (Sigma) as a substrate. All experiments were performed in triplicate. 10 μl of ChiA-FL (i.e. full length ChiA) (SEQ ID NO: 2), ChiA-NT (SEQ ID NO: 5), ChiA-CTD (SEQ ID NO: 40), ChiA-CTD^{E543M} (SEQ ID NO: 17), ChiA-CTD^{Q583}A (SEQ ID NO: 25) or ChiA-CTD^{Q617A} (SEQ ID NO: 27) at 10 μg/ml in PBS were mixed with 90 μl of substrate at 0.4 mg/ml dissolved in 20 mM sodium acetate pH 4.8. These samples and a 300 μl standard (50 μM ρ-nitrophenol, 100 mM sodium carbonate) were incubated at 37° C. for 30 min and then the ChiA reactions were quenched with the addition of 200 μl of 100 mM sodium carbonate. The release of the chromophore r-nitrophenol (pNP) was measured at 405 nm and ChiA samples were corrected for absorption in a control sample with added PBS instead of protein. 1 unit of activity per mg enzyme (U/mg) was defined as the release of 1 mmol of pNP/mg of ChiA/min.

Chitin Binding Assay 250 μl ChiA-FL (SEQ ID NO: 2), ChiA-NT (SEQ ID NO: 5), ChiA-N1 (SEQ ID NO: 7), ChiA-N2 (SEQ ID NO: 9), ChiA-N3 (SEQ ID NO: 11), ChiA-CTD (SEQ ID NO: 40), ChiA-CTD^{D504A} (SEQ ID NO: 13), ChiA-CTDH^{506A} (SEQ ID NO: 15), ChiA-CTD^{E543} (SEQ ID NO: 17), ChiA-CTD^{H544A} (SEQ ID NO: 19), ChiA-CTD^{N547A} (SEQ ID NO: 21), ChiA-CTD^{Q595A} (SEQ ID NO: 23) and BSA (Sigma) at 10 μM in 20 mM Tris-HCl pH 8.0, 200 mM NaCl were incubated with 50 μl chitin-resin (Sigma) and incubated whilst shaking for 30 min. The resin was washed three times with 500 μl of the same buffer and then proteins were eluted by incubating the resin in 250 μl of 8 M urea, 1% (w/v) SDS for 30 min whilst shaking. Protein samples prior to incubation with chitin-resin and the eluted protein/chitin-resin slurry were then analysed with SDS-PAGE. Eluted samples underwent an upward shift compared to the input samples due to the large differences in buffer conditions.

Crystal Structure Determination

Crystallization of ChiA CTD-domain (SEQ ID NO: 1) (30 mg/ml) was performed using the sitting-drop vapour-diffusion method grown in 0.2 M ammonium acetate, 0.1 M Bis-Tris pH 5.5, 45% (v/v) 2-Methyl-2,4-pentanediol at 293K. Native crystals were flash cooled in liquid nitrogen and diffraction data were collected at 100K on beamline I04 at the Diamond Light Source (DLS), UK. Crystals were also soaked for 1 min in well solution containing 1.0 M NaI, flash cooled in liquid nitrogen and data were collected at 100K on beamline I02 at the Diamond Light Source (DLS), UK. Data were processed with the CCP4 suite of programs. The structure of ChiA CTD-domain was determined with I-SIRAS. Models were built and refined with the CCP4 suite of programs.

SAXS Data Collection and Analysis

SAXS data were collected on beamline B21 at the Diamond Light Source (DLS), UK at 20° C. Full-length ChiA (SEQ ID NO: 2) in 20 mM Tris-HCI pH 8, 200 mM NaCl were measured at 4, 2, 1 and 0.5 mg/ml concentrations, after gel filtration using a Superdex 200 column (GE Healthcare), over a momentum transfer range of 0.004<q<0.4 Å-1. A fresh sample of BSA was measured as a standard. Buffer subtraction, intensity normalization, and data merging for the different sample concentrations were performed in SCATTER (DLS, UK). ChiA data collected above 1 mg/ml showed signs of aggregation and were discarded. Further analysis was carried out with the 1 mg/ml data using a q range 0.008<q<0.2 Å-1. Data was processed using the ATSAS software. An initial model of ChiA was created from PHYRE2 models of the N1- and N3-domains (residues 7-132; 300-399), a ROBETTA model of the N2-domain (residues 138-290) (SEQ ID NO: 9) and our crystal structure of the C-domain (residues 424-762) (SEQ ID NO: 1), with domain linker sequences kept unstructured. Determination of molecular model ensembles that best fit the SAXS data was performed using EOM2.0 software.

Mucin Binding ELISA

Immulon 2-HB 96-well plates (VWR) were coated overnight at 4° C. with 50 μl of partially purified mucins from bovine submaxillary glands (type I-S; Sigma) and porcine stomachs (type II and III; Sigma) at 100 pg/ml in 50 mM Carbonate/Bicarbonate pH 9.6. Wells were blocked for 1 hr at 25° C. with 200 μl of 0.1% (w/v) bovine serum albumin (BSA) in PBS-0.05% Tween 20 and then washed once with 200 μl of incubation buffer (0.05% (w/v) BSA in PBS-0.05% Tween 20). Wells were then incubated for 3 hrs at 25° C. with 50 μl of ChiA-FL (SEQ ID NO: 2), ChiA-NT (SEQ ID NO: 5), ChiA-N1 (SEQ ID NO: 7), ChiA-N2 (SEQ ID NO: 9), ChiA-N3 (SEQ ID NO: 11), ChiA-CTD (SEQ ID NO: 40), ChiA-CTDD504A (SEQ ID NO: 13), ChiA-CTDH506A (SEQ ID NO: 15), ChiA-CTDE543M (SEQ ID NO: 17), ChiA-CTDH544A (SEQ ID NO: 19), ChiA-CTDN547A (SEQ ID NO: 21), ChiA-CTDQ583A (SEQ ID NO: 25), ChiA-CTDQ595A (SEQ ID NO: 23), ChiA-CTDQ617A (SEQ ID NO: 27), NttE and SsIE at 10 μM in incubation buffer. This was followed by four washes with 200 μl of incubation buffer and incubation with 50 μl of anti-His-HRP antibody (Sigma), diluted 1:2000 in incubation buffer for 1 hr at 24° C. After four washes with 200 μl of incubation buffer, 150 μl of o-Phenylenediamine dihydrochloride (Sigma) was added for 30 min and then data was recorded at 450 nm.

Immunoblot for Detecting Secreted Mucinase Activity

L. pneumophila strains that had been grown for three days on BCYE agar were suspended into 20 ml of BYE broth to an OD660 of 0.3 and grown overnight at 37° C. to an OD660 of 3.0-3.3. Bacteria were sub-cultured into fresh BYE medium to an OD660=0.3 and grown, with shaking, to an OD660 of 1.0, which corresponded to the mid-log phase. Supernatants were collected, filtered through a 0.22-μm filter, and concentrated using 10-kDa Amicon concentrators (EMD Millipore). 200 μl of concentrated supernatants were incubated with 200 or 400 μg of type II porcine stomach mucins. As controls, the mucins were either incubated in uninoculated BYE broth or in BYE broth containing 50 pl of a known mucinase cocktail, which consisted of 10 μl each of pepsin (0.5 mg/ml), pronase (10 mg/ml), β-N-acetylglucosaminidase (2.5 μM), fucosidase (5 U/ml), and DTT (1 mM) dissolved in 940 μl of ddH20. The various samples were incubated statically for 3 h at 25° C. and then subjected to electrophoresis prior to immunoblotting. Reactions were stopped by adding 200 μl of 2× Laemmli buffer and incubating for 5 min at 100° C., and 35 μl of each sample was electrophoresed through a Criterion 4-20% SDS-PAGE gel (Bio-Rad) for 1.5 h at 250 volts. The separated reaction products were transferred onto PVDF membrane over the course of 13 min using the semi-dry Invitrogen Power-Blotter and Power Blotter transfer blotting solution. Following incubation in 1% BSA in TBST for 1 h at room temperature, the membranes were incubated overnight at 4° C. with biotinylated wheat germ agglutinin that had been diluted 1:2000 (from a 1 mg/ml stock) in TBST with BSA. After three, 5-min washes with TBST buffer, the membranes were further incubated for 1 h at 37° C. with Avidin-HRP that had been diluted 1:2000 in BSA-containing TBST. Finally, subsequent to a series of washes, the blot was incubated for 1 min in 2 ml Amersham ECL reagent and then exposed to X-ray film.

Mucin Coated Transwell Penetration Assay

L. pneumophila wild-type 130b (WT) and chiA mutant NU318 (chiA) were grown for three days on BCYE agar and then resuspended into 20 ml of BYE broth to an OD660 of 0.3 and grown overnight at 37° C. to an OD660 of 3.0-3.3. Bacteria were sub-cultured into fresh BYE medium to an OD660 of 0.3 and grown, with shaking, to an OD660 of 1.0, which corresponded to the mid-log phase. Bacteria were then diluted in BYE broth to OD660 of 0.3. Transwell plates (Corning) were used for analysis of bacterial crossing of a mucin layer. 500 μl of sterile BYE broth was added to the bottom of either empty wells, or wells containing 3.0 μm transwells. Transwells were either kept uncoated or coated with 50 or 100 μg of type II porcine mucin in bicarbonate buffer. After 1 hr of coating transwells with either control BYE broth or type II mucin, 500 μl of 0.3 OD660 L. pneumophila (chiA or WT) was applied to either the empty well, or to the top of a transwell. Bacteria that were able to cross the transwell membrane to the bottom of the well were collected 2 hrs after application to wells, diluted and plated onto BCYE plates for CFU analysis. Each experiment had 3 technical replicates. N=3 experimental replicates were analyzed. Two-way ANOVA with Boneferri post-hoc statistical analysis was used.

Immunoblot for Detecting Recombinant ChiA MUC5AC Activity

Porcine type II stomach mucin (Sigma) was dissolved in PBS at 8 mg/ml and incubated for 5 min with 5 mM EDTA to remove divalent cations. This was then buffer exchanged into PBS using a 30-50 kDa MWCO concentrator (Generon). Recombinant ChiA-FL (SEQ ID NO: 2), ChiA-NT (SEQ ID NO: 5), ChiA-CTD (SEQ ID NO: 40), ChiA-CTD^{D504A} (SEQ ID NO: 13), ChiA-CTD^{H506A} (SEQ ID NO: 15), ChiA-CTD^{E543M} (SEQ ID NO: 17), ChiA-CTD^{H544A} (SEQ ID NO: 19), ChiA-CTD^{N547A} (SEQ ID NO: 21), ChiA-CTD^{Q583A} (SEQ ID NO: 25), ChiA-CTD^{Q595A} (SEQ ID NO: 23), ChiA-CTD^{Q617A} (SEQ ID NO: 27) and SsIE were incubated for 5 min with 5 mM EDTA to remove any bound metal ions. These were then dialyzed extensively against PBS with 1 mM ZnCl₂ and the concentrations adjusted to 20 μM. Mucins were mixed with an equal volume of protein in either PBS, 1 mM ZnCl₂ or PBS, 5 mM EDTA and incubated for 3 hr at 25° C. Reactions were stopped with the addition of an equal volume of 2× Laemmli buffer and incubated for 5 min at 100° C. Samples were then run on a Criterion 4-20% SDS-PAGE gel (Bio-Rad), followed by transfer onto a PVDF membrane using the semi-dry Invitrogen Power-Blotter and Power Blotter transfer blotting solution. The membrane was incubated in 1% BSA in TBST for 1 h at room temperature, and then overnight at 4° C. with biotin conjugated MUC5AC antibody (Thermo Fisher Scientific) that had been diluted 1:2000 in TBST with BSA. After three, 5-min washes with TBST buffer, membranes were incubated for 1 h at 37° C. with Avidin-HRP diluted 1:2000 in BSA-containing TBST and followed by three washes for 5-min each. This was then incubated with avidin-HRP (1:2000 dilution) for 1 hr at 25° C. and then treated with enhanced chemiluminescence substrate (ECL; Pierce) before detection by enhanced chemiluminescence.

Molecular Dynamics

MD simulations and analyses were performed using GROMACS 2016 v3 software. The protein was described using the Amber99SB*-ILDN force field and solvated using a truncated octahedral box of TIP3P water molecules. A minimal distance of 12 ∈ was set between the protein and the walls of the box. The charge of the ionisable residues was set to that of their standard protonation state at pH 7. Zn²⁺ ions were added by randomly replacing water molecules. A high Zn²⁺ concentration (0.75 M) was used to have a faster sampling of possible Zn²⁺ sites around the protein surface. Cl⁻ counterions were added to neutralise the system.

Periodic boundary conditions were applied. The equations of motion were integrated using the leap-frog method with a 2-fs time step. The LINCS algorithm was chosen to constrain all covalent bonds in the protein, while SETTLE was used for water molecules. The Particle Mesh Ewald (PME) method was used for electrostatic interactions, with a 9-Å cut-off for the direct space sums, a 1.2-Å FFT grid spacing, and a 4-order interpolation polynomial for the reciprocal space sums. A 9-Å cut-off was used for van der Waals interactions. Long-range corrections to the dispersion energy were included.

Each system was minimised through 3 stages with 2000 (positional restraints on heavy atoms)+3000 steps of steepest descent, followed by 2000 steps of conjugate gradient. Positional restraints on heavy atoms were initially set to 4.8 kcal/mol/Å² and they were gradually decreased to 0 in 1.5 ns, while the temperature was increased from 200 to 300 K at constant volume. The system was then allowed to move freely and was subjected to 1-ns equilibration in NVT conditions at T=300 K. This was followed by a 2-ns equilibration in NPT conditions with T=300 K and p=1 bar. For these equilibration steps, the Berendsen algorithm was used for both temperature and pressure regulation with coupling constants of 0.2 and 1 ps, respectively. At last, a 2-ns NPT equilibration was run after switching to the v-rescale thermostat with a coupling constant of 0.1 ps and the Parrinello-Rahman barostat with a coupling constant of 2 ps. Production NPT runs were then performed for 50 ns, saving the coordinates every 1 ps. Multiple replicas were run, with each replica starting from a different configuration of the ions around the protein, for an aggregated simulation time of 1.7 ps (34×50 ns). The spatial distribution function (sdf) of Zn²⁺ around the protein was calculated with the gmx spatial tool from GROMACS. Trajectories from the different replicas were first concatenated together and each frame was aligned through a best-fit superposition to the starting frame using the protein coordinates. A 0.5-Å grid spacing was used for the sdf calculation. The average of non-null sdf values was calculated and isosurfaces connecting points with sdf=20, 25 and 30× average sdf were considered.

Isothermal Calorimetry

ITC experiments were performed at 293 K using a MicroCal iTC200 calorimeter (Malvern). ChiA-CTD (SEQ ID NO: 40), ChiA-CTD^{E543M} (SEQ ID NO: 17), ChiA-CTD^{E543M/D504A} (SEQ ID NO: 29), ChiA-CTD^{E543M/H506A} (SEQ ID NO: 31), ChiA-CTD^{E543M/H544A} (SEQ ID NO: 33), ChiA-CTD^{N547A} (SEQ ID NO: 21) and ChiA-CTD^{Q595A} (SEQ ID NO: 23) were dialyzed into buffer containing 20 mM Tris pH 8.0, 200 mM NaCl. Experiments were performed by placing the solution containing ChiA proteins in the cell at 70 μM and the solution containing the zinc (dissolved in dialysis buffer) in the syringe at 2 mM. For each titration 18 injections of 2 μl were performed. Integrated data, corrected for heats of dilution, were fitted using a nonlinear least-squares algorithm to a 1:1 binding curve, using the MicroCal Origin 7.0 software package. Each experiment was repeated at least twice, and representative values are reported.

Results

ChiA is a Multi-Domain Protein

Full-length ChiA from L. pneumophila 130b (ChiA-FL; numbered 1-762 for the mature protein; NCBI accession WP_072401826.1) with an N-terminal His6-tag was produced in Escherichia coli K12 and purified by nickel-affinity and size exclusion chromatography. Despite extensive screening, ChiA-FL resisted crystallization and therefore bioinformatics analysis was used to produce a series of subdomain constructs for further characterization (FIG. 1A). While previous examination of the C-terminal domain of ChiA (ChiA-CTD: residues 419-762) had revealed high primary sequence homology to other GH18 chitinase domains (7), the N-terminal region (ChiA-NT; residues 1-417) contains unique primary sequence with no significant homology to any other known protein. Nonetheless, through secondary structure prediction and template based modelling using the Phyre2 and Robetta servers, three putative N-terminal subdomains were identified based on predicted structural similarity with carbohydrate-binding modules (CBMs; ChiA-N1: residues 1-140), fibronectin type-III-like domains (Fn3; ChiA-N2: residues 138-299) and a chitinase A N-terminal domain (ChiN; ChiA-N3: residues 285-417) (FIG. 1A).

To examine the function of the ChiA N-terminal domains their endochitinase activity was examined. Each construct was expressed with an N-terminal His6-tag in E. coli K12 and purified by nickel-affinity and size exclusion chromatography. All reagents were well folded and ChiA-FL and ChiA-CTD were both active against p-nitrophenyl β-D-128 N,N′,N″ triacetylchitotriose (pNP-[GlcNAc]₃) but no activity was detected for ChiA-NT or an E543M ChiA-CTD chitinase active site mutant (ChiA-CTD^{E543M}) (FIG. 1B). Binding of ChiA sub-domains to immobilized chitin was assayed and it was observed that in addition to ChiA-CTD, the N1-domain also recognizes chitin polymers, which supports its role as a carbohydrate binding module (FIG. 1C).

Atomic Structure of ChiA-CTD

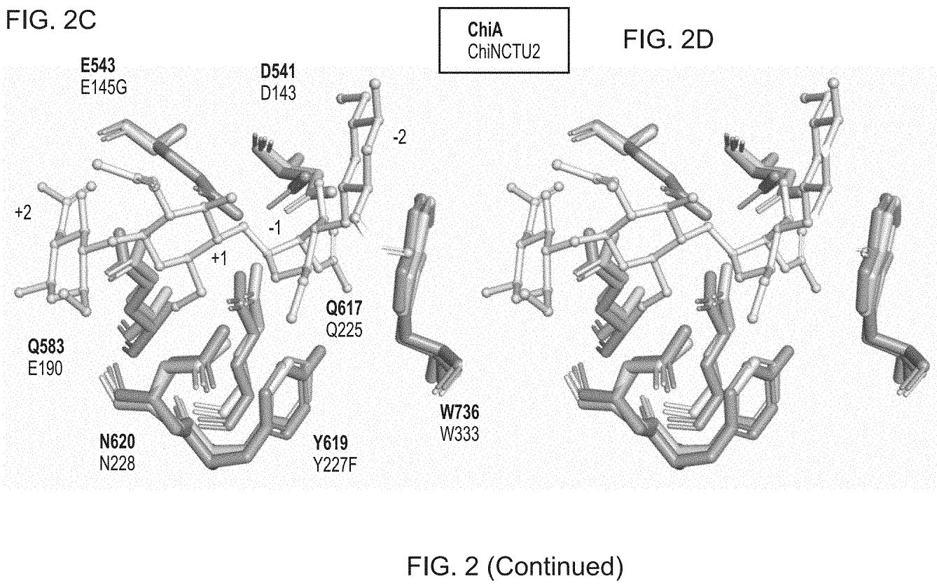

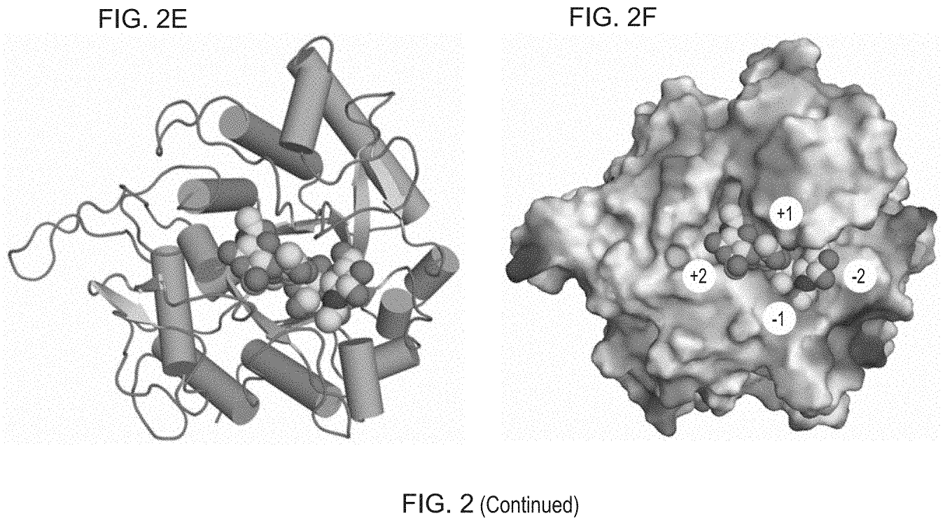

Crystallographic studies of the ChiA subdomains were then initiated. Crystals for ChiA-CTD were obtained and its structure was determined using iodide single isomorphous replacement with anomalous scattering (SIRAS) phasing. Electron-density maps were refined to 1.7 Å and the final model contains two identical chains, with all molecules built except for the N-terminal His6-tags and adjacent ChiA-CTD residues Val419 to Gly424. This indicates that residues 419 to 423 of the ChiA sequence may be deleted without losing protein function of ChiA-CTD, resulting in the sequence of SEQ ID NO: 1. Also indicated is that these five residues are not included in the crystal because they stay unstructured during crystallisation. Each chain forms an anticipated GH18 α/β-fold and is composed of 11 β-strands and 13 α-helices (FIG. 2A, 2B).

The overall structure of ChiA-CTD is highly similar to other GH18 chitinase domains, and the Dali server identified Bacillus cereus ChiNCTU2 enzyme inactive E145G/Y227F mutant in complex with chitotetraose (Protein Data Bank (PDB) ID code 3n18) as having the highest homology (Z score: 36.3; rmsd: 2.2 Å). The chitinase active sites of ChiNCTU2 and ChiA-CTD have high primary sequence identity and tertiary structure homology (FIG. 2C, D) and modelling of chitotetraose binding indicates that chitin lines a negatively charged valley on the surface of ChiA-CTD (FIG. 2E, F). In this binding model, Gln583 and Gln617 have a central role in the correct positioning of chitotetraose in the ChiA-CTD active site. Therefore Q583A and Q617A mutants in recombinant ChiA-CTD were created (SEQ ID NO: 25/26 and SEQ ID NO: 27/28 respectively), and as anticipated, these mutants showed no activity against pNP-[GlcNAc]₃ (FIG. 1B). However, L. pneumophila ChiA-CTD also possesses unique features that are not observed in homologous structures. These include an additional α-helix (α3), an extended β3-α3 loop, an extended α6-α6′ loop and an extended β7-α7 loop (FIG. 2A, 2B).

ChiA is an Elongated and Dynamic Structure in Solution

Small angle X-ray scattering (SAXS) was used to model the global structure of full-length ChiA in solution. Four different concentrations at 4, 2, 1, and 0.5 mg/ml were measured but signs of aggregation were apparent at concentrations above 1 mg/ml. All further analysis was carried out with the data from the 1 mg/ml sample. Guinier analysis suggested a radius of gyration (R_{g}), the root mean square distance to the particles centre of mass, of 5.43 nm and analysis of the distance distribution function (P(r)) suggested a maximum particle dimension (Dₘₐₓ) of 17.77 nm and R_{g} of 5.45 nm. Using BSA as a standard, a particle molecular mass of 89.2 kDa was calculated, which is within the method error range for a monomeric 82.6 kDa ChiA.

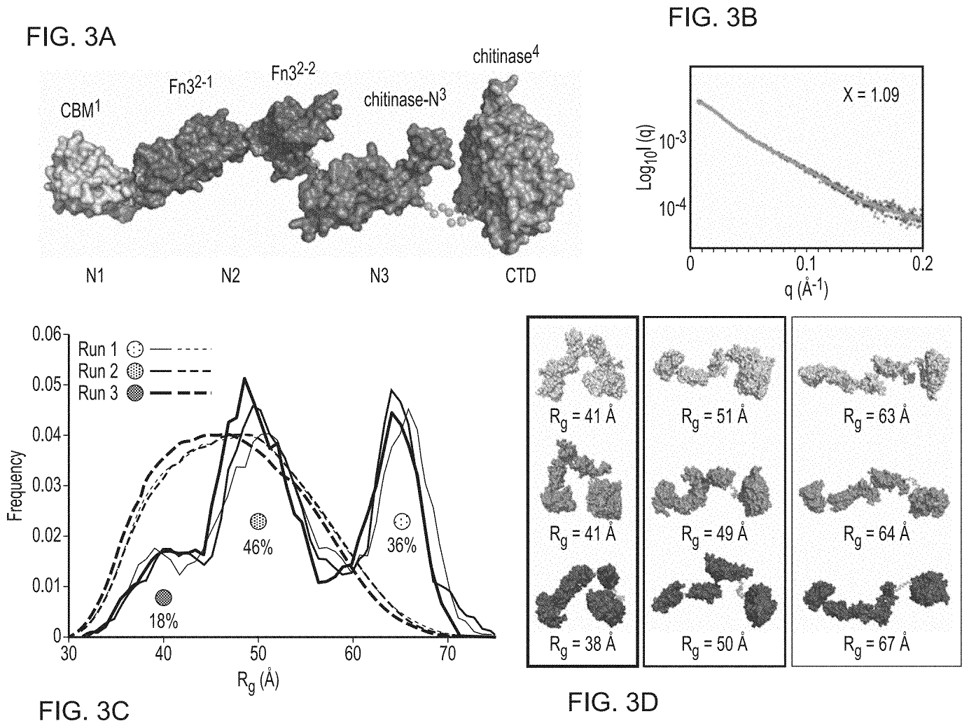

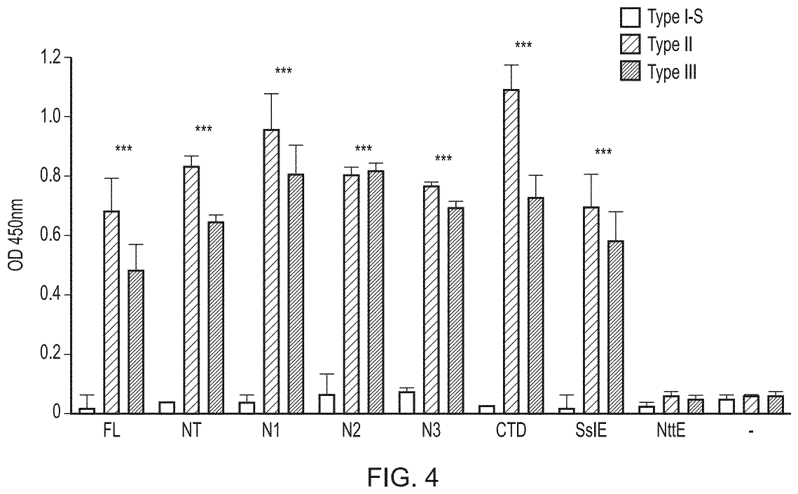

The SAXS data indicated that ChiA is a highly dynamic particle in solution and this is likely due to flexibility within the ChiA inter-domain linkers. Therefore ensemble optimization method (EOM) was used to determine molecular model ensembles of ChiA that best fit the SAXS data. As it was not possible to obtain crystals for ChiA N-domains, an initial model of ChiA was created using a Phyre2 derived N1-domain (residues 22-147), a Robetta derived N2-domain (encompassing two further subdomains: residues 152-245 and 248-305), a Phyre2 derived ChiA N3-domain (residues 315-414) and the crystal structure of ChiA-CTD (residues 439-777), separated by flexible linkers (FIG. 3A). Ensemble optimization analysis of the scattering data yielded an excellent fit between experimental and calculated SAXS profiles (χ²:1.1), which again indicates that ChiA is highly flexible in solution (R_{flex} 91.4) with conformation ensembles clustered within three populations (FIG. 3B, 3C, 3D). The majority of the simulated conformations exhibited partially extended or fully extended structures at R_{g} 44-56 Å or R_{g} 60-71 Å, respectively, whereas minor conformations of closed structures were also populated at R_{g} 33-43 A. This analysis provides experiential support for the global features of our ChiA N-domain models but also suggests that there is cooperation between ChiA domains. As inter-domain flexibility is a key feature of processive enzymes, these data suggest that processivity may also be important for the function of ChiA.

ChiA is a Mucin Binding Protein

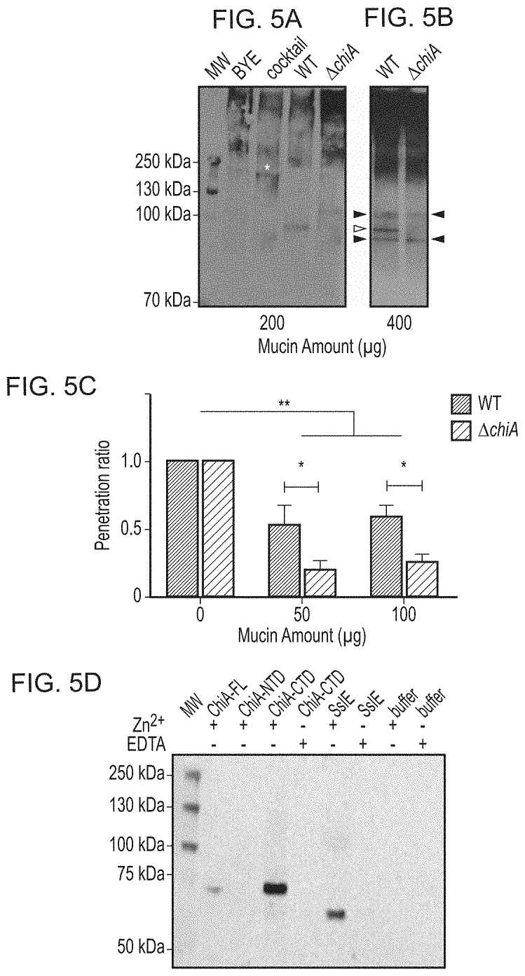

Some bacterial chitinases and chitin binding proteins are able to promote infection through adhesion to and/or degradation of host glycoconjugates (8) and it was hypothesized that ChiA may interact with exogenous mucins in the lungs and elsewhere. Therefore the binding capacity of recombinant ChiA-FL, ChiA domains, L. pneumophila NttE, another T2SS substrate (negative control) and E. coli SsIE, a known mucin binding protein and mucinase (9), to immobilized commercially available mucin extracts was examined by ELISA using anti-His antibodies (FIG. 4). All ChiA samples and SsIE displayed significant adhesion to mucins isolated from porcine stomachs (type II and III), but this was not observed with a mucin extract from bovine submaxillary glands (type I-S). Conversely, NttE showed no binding to any of the mucin samples. This confirmed that ChiA has additional specificity for non-chitinous ligands.

ChiA Increases Penetration of L. pneumophila through the Mucin Layer

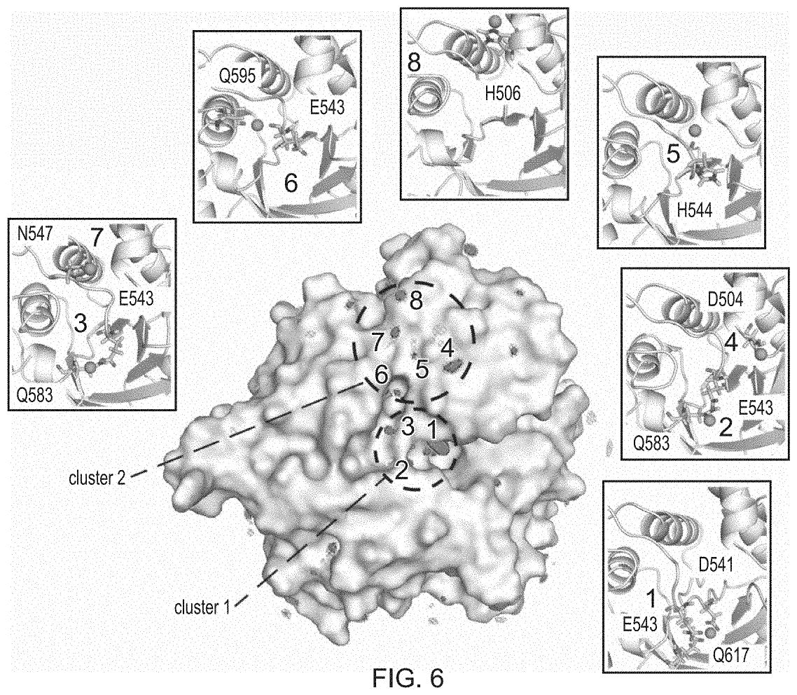

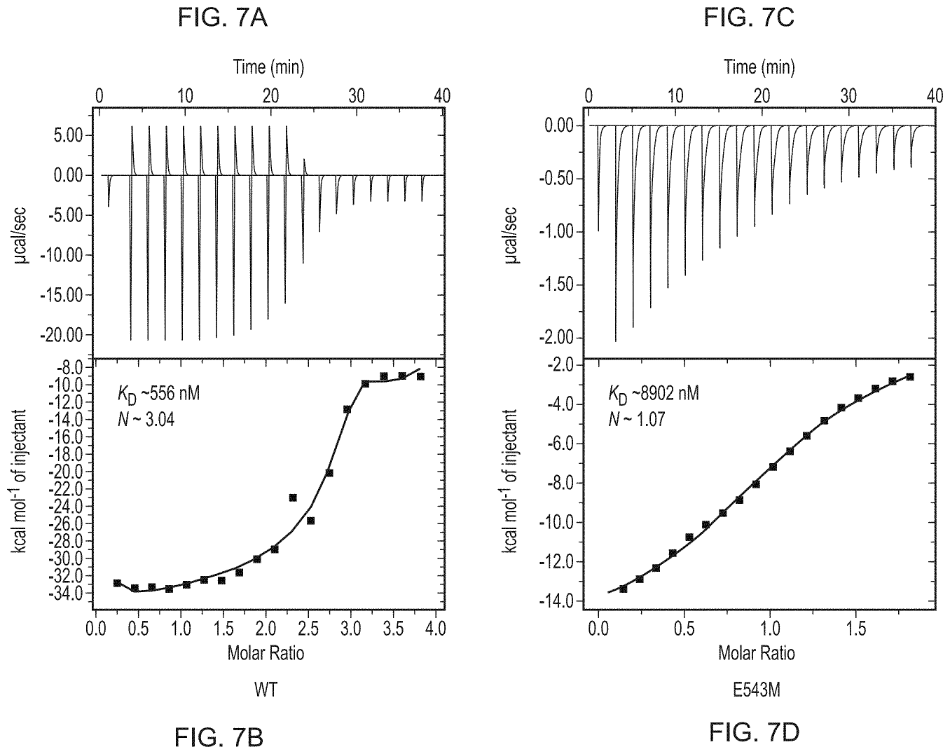

It was next examined whether secreted ChiA is able to degrade mucins. Porcine stomach type II mucin extract was incubated with supernatants from L. pneumophila 130b wild-type and NU318 (chiA) mutant strains or a cocktail of enzymes (pepsin, pronase, β-N-acetylglucosaminidase, fucosidase) with known activity against mucins, and then analysed by immunoblotting using wheat germ agglutinin (FIG. 5A). While the majority of the mucin extract ran at >500 kDa, after incubation with the mucinase cocktail there was a reduction in high molecular weight species and the appearance of a new band at ˜200 kDa. When the extract was incubated with L. pneumophila 130b wild-type supernatant there was again a reduction of high molecular weight material but with the addition of a new fragment at ˜95 kDa. On the other hand, the chiA mutant supernatant produced a profile that was more similar to the control, although there was evidence of another new species at ˜100 kDa. When the experiment was performed using a greater amount of mucin, the difference between the wild-type and mutant was even more evident (FIG. 5B). Three clear fragments (˜100 kDa, ˜95 kDa and ˜90 kDa) could be observed in the type II mucin extract incubated with wild-type supernatants, with the middle band absent when incubated with the chiA mutant supernatant. This band is dependent upon ChiA and implies that ChiA can function as both chitinase and a mucinase.

Mucins are high molecular weight glycoproteins that contain large numbers of heavily O-glycosylated serine/threonine rich repeat sequences (10). They exist as cell surface exposed transmembrane proteins or secreted gel-forming proteins of the mucosal barrier and act as a first line of defence against bacterial infection. The normal stomach mucosa is characterised by expression of MUC1, MUC5AC, and MUC6 mucins, however, MUC1 and MUC5AC are also major mucins expressed in the mammalian airway and lung. Therefore, to determine whether ChiA can facilitate mucin penetration of L. pneumophila an artificial mucin penetration assay was performed. After a 2 hr incubation with either L. pneumophila wild-type 130b or chiA mutant NU318, it was observed that there was a 2.7- and 2.4-fold decrease in the number of colonies from the chiA mutant compared with wild-type in the presence of 50 and 100 μg type II mucin extract, respectively (FIG. 5C). This confirms that ChiA promotes L. pneumophila dissemination through mucin layers.

ChiA-CTD is a Zn2⁺-Dependent Peptidase

The ability of recombinant ChiA to specifically degrade MUC5AC within type II mucin extracts was then examined by immunoblotting and compared its profile to that of recombinant E. coli SsIE. Intact MUC5AC did not enter the gel in the buffer controls and this was likely due to its high carbohydrate content and large mass (>500 kDa before glycosylation). However, incubation of type II mucin extract with ChiA-FL and ChiA-CTD, but not ChiA-NT, resulted in the processing of MUC5AC into a new ˜70 kDa fragment (FIG. 5D). When the mucin extract was incubated with SsIE, MUC5AC was processed into a different ˜60 kDa species. SsIE is a member of the M60 family of metalloproteases, which use a HExxH motif to coordinate Zn²⁺ in their active sites. In the presence of the metal chelating agent ethylenediaminetetraacetic acid (EDTA) no activity was detected for SsIE or ChiA-CTD and this clearly shows that the C-terminal domain of ChiA has dual enzymatic activity. This also suggests that ChiA and SsIE use a similar peptidase mechanism for the degradation of mucins.

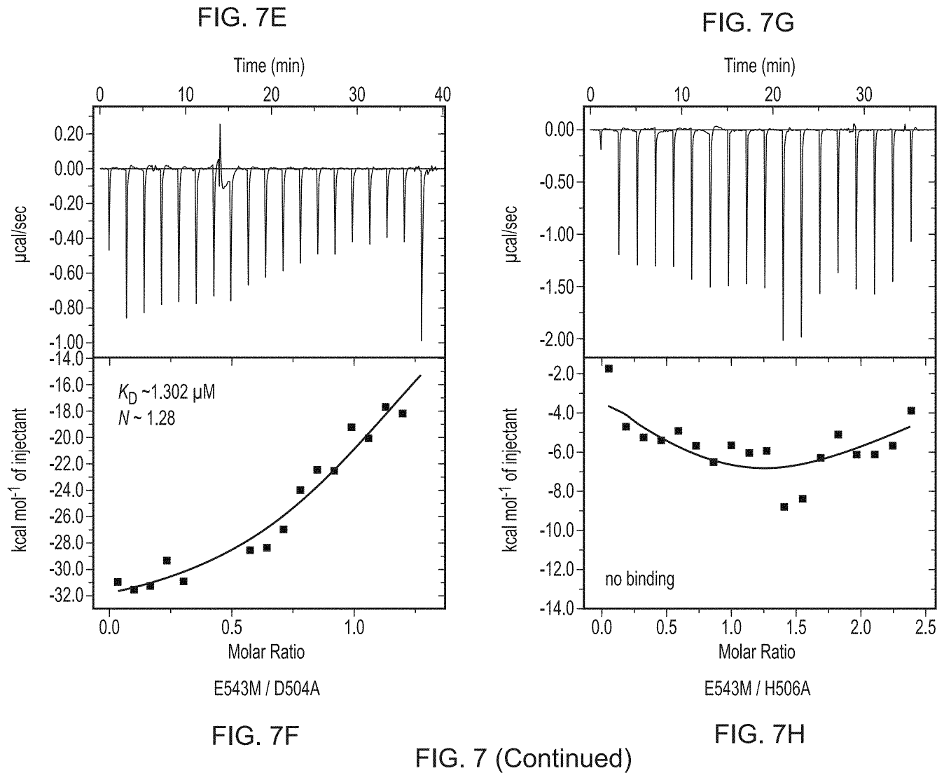

To evaluate this further Molecular Dynamics (MD) simulations were performed and examined the ability of ChiA-CTD to bind Zn²⁺ in silico. The protein was ‘soaked’ in a water solution at high Zn²⁺ concentration and the system was then left to evolve over time to identify the regions on the protein surface where Zn²⁺ ions tend to bind. Multiple short simulations were run starting from different random placements of Zn²⁺ ions, for an aggregated simulation time of 1.7 μs. Analysis of the Zn²⁺ spatial distribution function (sdf) calculated on the concatenated trajectories highlighted multiple high Zn²⁺ density sites in the region around the chitinase active site, providing information on the different ways in which Zn²⁺ could bind to the protein in this region. The highest density was found at the chitinase active site (region 1), where Zn²⁺ is coordinated by Asp541, Glu543 and Gln617, with two other sites in close proximity (regions 2,3) coordinated by Glu543 and Q583 (FIG. 6). Binding of two Zn²⁺ ions in the active site of Bacillus cereus ChiNCTU2 has been shown to inhibit chitinase activity and indicates that metal binding could modulate the different enzyme activities in ChiA (13). A unique cluster of Zn²⁺ sites was also located away from the chitinase active site, involving residues Asp504 (region 4), His544 (region 5), Glu543 and Gln595 (region 6), Asn547 (region 7) and His506 (region 8) (FIG. 6).

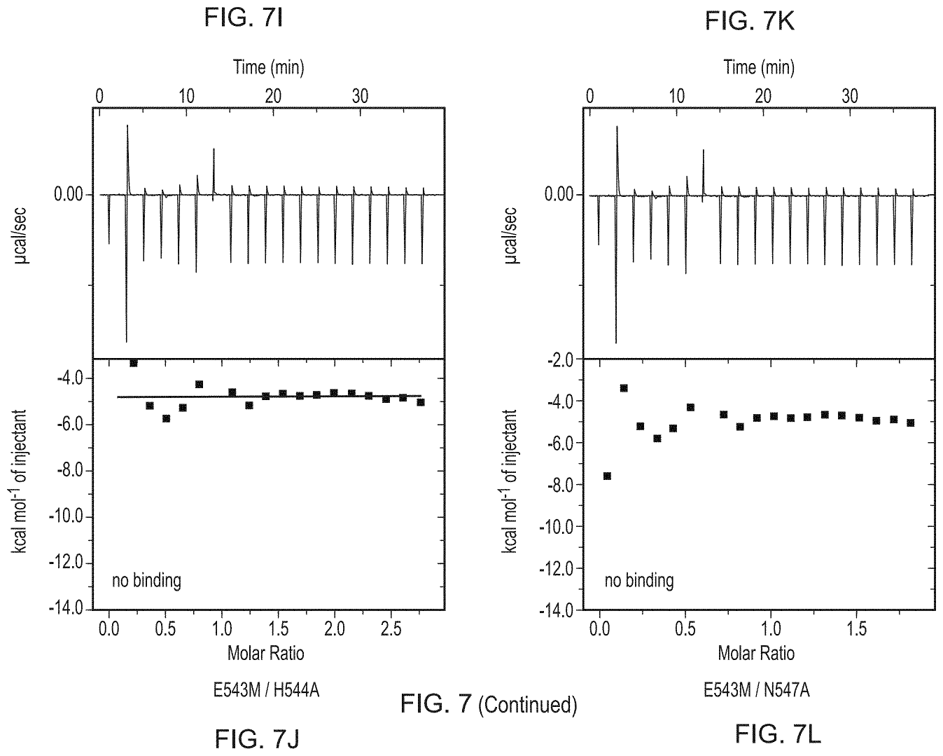

To verify these in silico observation isothermal titration calorimetry (ITC) was used and measured an equilibrium dissociation constant (KD) of 556 nM for approximately three Zn²⁺ ions (N=3.04) binding to wild-type ChiA-CTD (SEQ ID NO: 40) (FIG. 7A, 7B). During the experiment both exothermic and endothermic signals were observed, and as these were not detected in a reverse titration and this may reflect a different binding mechanism for each site. Binding of Zn²⁺ to ChiA-CTD^{E543M} was then examined, and exothermic binding was observed with a ˜1.6-fold reduction in affinity (KD 890 nM) at a single site (N=1.07) (FIG. 7C, 7D). This indicated that Glu543 is involved in the coordination of zinc in the chitinase active site. To assess whether residues from the second cluster also formed a genuine binding site, E543M/D504A (SEQ ID NO: 29), E543M/H506A (SEQ ID NO: 31), E543M/H544A (SEQ ID NO: 33), E543M/N547A (SEQ ID NO: 35) and E543M/Q595A (SEQ ID NO: 37) double mutants were created in ChiA-CTD. When Zn²⁺ binding to ChiA-CTD^{E543M/D504A} (SEQ ID NO: 29) was assessed using ITC exothermic binding was measured, with a further ˜1.5-fold reduction in affinity (KD 1.3 μM) at a single site (N=1.28) (FIG. 7E, 7F). However, examination of Zn²⁺ binding to ChiA-CTD^{E543M/H506A} (SEQ ID NO: 31) (FIG. 7G, 7H), ChiA-CTD^{E543M/H544A} (SEQ ID NO: 33) (FIG. 7I, 7J), ChiA-CTD^{E543M/N547A} (SEQ ID NO: 35) (FIG. 7K, 7L) and ChiA-CTD^{E543M/DQ595A} (SEQ ID NO: 37) (FIG. 7M, 7N) showed no binding. This indicates that His506, His544, Asn547 and Q595 form a unique zinc binding site in ChiA and are therefore significant for providing mucolytic effect.

ChiA-CTD Uses a Novel Mechanism to Cleave Mucin-Like Glycoproteins

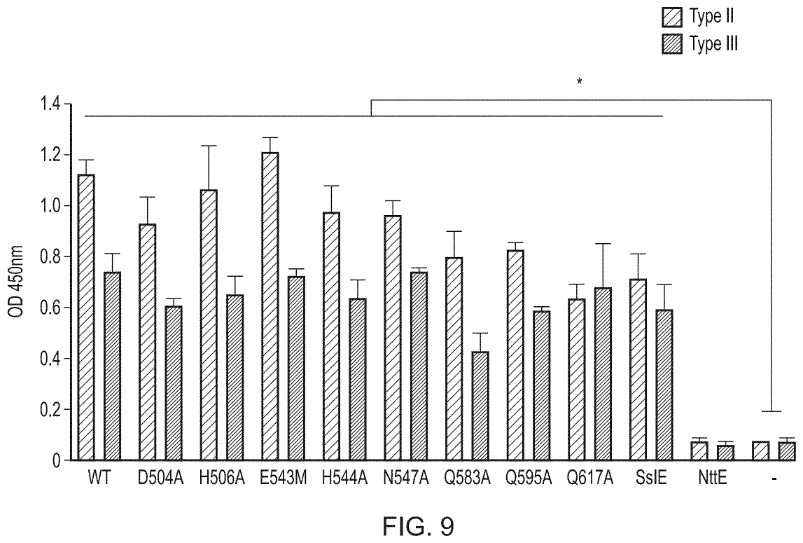

To assess the role of these residues in the degradation of MUC5AC, D504A (SEQ ID NO: 13), H506A (SEQ ID NO: 15), H544A (SEQ ID NO: 19), N547A (SEQ ID NO: 21) and Q595A (SEQ ID NO: 23) single site mutations were created in ChiA-CTD. All constructs were well folded and with our existing single site ChiA variants (ChiA-CTD^{E543M} (SEQ ID NO: 17), ChiA-CTD^{Q583A} (SEQ ID NO: 25) and CTDQ^{617A} (SEQ ID NO: 27)), they were still able to bind immobilized type II and III mucin extracts in ELISA assays (FIG. 9). These proteins were then incubated with type II mucin extract and inspected their MUC5AC degradation profiles by immunoblotting (FIG. 8A). Incubation with ChiA-CTD (SEQ ID NO: 40) and ChiA-CTDE543M (SEQ ID NO: 17) both produced identical MUC5AC degradation patterns, while all other ChiA-CTD variants showed no activity. This demonstrates that ChiA-CTD (SEQ ID NO: 40) has broad specificity for mucin-like glycoproteins and its peptidase activity is independent from the adjacent chitinase active site. In view of the above, we show (FIG. 8B, 8C) that these H506, H544 and N547, Q595 as well as D504 are significant for providing mucolytic activity. It appears that D504 is in the active site of the enzyme and is important as a general base during catalysis.

Discussion

Chitin is highly abundant in the environment and can function as a source of carbon and nitrogen but several chitinases have been identified as key virulence factors in bacterial disease (8). These include Enterococcus faecalis efChiA, E. coli ChiA, Vibrio cholerae ChiA2, Francisella tularensis ChiA, Listeria monocytogenes ChiA and ChiB, Pseudomonas aeruginosa ChiC, Salmonella Typhimurium ChiA and L. pneumophila ChiA. Although it is unclear how these enzymes perform these dual functions, there is strong evidence that they interact with host glycoconjugates and through their localization and/or enzymatic activity are able to modulate host defence mechanisms (8). It has been determined that L. pneumophila ChiA has activity against porcine stomach derived mucins, and the degradation of MUC5AC produces a similar degradation profile to the M60-family E. coli zinc-aminopeptidase SsIE (9). Recent structural analysis of Bacteroides thetaiotaomicron BT4244, Pseudomonas aeruginosa IMPa and Clostridium perfringens ZmpB M60 proteins has revealed unique structural adaptations that allow them to accommodate different glycan sequences while all cleaving the peptide bond immediately preceding the glycosylated residue (13). Similarly, E. coli StcE is an M66-family zinc metalloprotease that recognizes distinct peptide and glycan motifs in mucin-like proteins and then cleaves the peptide backbone using an extended HExxHxxGxxH motif (15). In StcE, three histidine residues in the conserved motif act as ligands for a single catalytic zinc, while in M60 enzymes two histidine and another residue perform this role. A nucleophilic water molecule is the fourth ligand for the zinc, and this is coordinated by a conserved glutamate, which acts as a general base during catalysis. It has been shown that L. pneumophila ChiA functions in a similar fashion to SsIE and StcE but as it does not contain a HExxH motif, ChiA represents a new class of peptidase that can degrade mammalian mucin-like proteins via a novel mechanism.

Four residues in ChiA that are significant for providing zinc binding in the peptidase active site have been identified (His506, His544, Asn547 and Gln595) and these likely coordinate a single zinc ion. These residues along with Asp504, Gln583 and Gln617 are also significant for providing peptidase activity. Examination of the ChiA-CTD structure suggests that His544, Asn547 and Gln595 are ligands for the zinc, with Asp504 the general base (FIG. 8B, 8C). His506 packs against the N547 and H544 loop and may have a role in correctly structuring this region of ChiA-CTD. Gln583 and Gln617 have important roles in the optimal positioning of chitin for processing within the chitinase active site (FIG. 2C, 2D). Lack of peptidase activity in ChiA-CTD^{Q583A} (SEQ ID NO: 25) and ChiA-CTD^{Q617A} (SEQ ID NO: 27) suggests that these residues are also involved in binding glycan motifs in mucin-like proteins. However, sequence alignment of L. pneumophila ChiA with other virulent bacterial chitinases (8), including the mucin degrading V. cholerae ChiA2, does not show conservation of these residues and modelling of their tertiary structures using the Phyre2 server also highlights significant differences within their chitin binding surfaces. This implies that other virulent bacterial chitinases either promote pathogenesis using an alternative mechanism or that the specific location of the peptidase active site is unique to each enzyme and shapes their glycan specificity and function.

Mucins derived from the lung are not commercially available, but it has been shown that ChiA has specificity for and can degrade MUC5AC purified from the porcine stomach, which is also a major mucin expressed in the human airway and lung [2,4]. MUC5AC is composed of T-antigen (Galβ1-3GalNAcαSer/Thr), core 2 (GlcNAcβ1-6(Galβ1-3)GalNAcαSer/Thr) and sialyl T-antigen (NeuAcα2-6(Galβ1-3)GalNAcαSer/Thr) core glycan structures (12). While the T-antigen contains a linear array of carbohydrates, core 2 and sialyl T-antigen have branched structures. Our study suggests that the specificity of ChiA-CTD for O-glycosylated substrates is mediated by glycan recognition in the chitin binding groove, which then orientates the peptide backbone of the substrate towards the peptidase active site for proteolysis (FIG. 8B, 8C).

MUC5AC is a gel forming mucin and the mucin penetration assay indicates that one role for ChiA in the lung is to facilitate bacterial penetration of the alveolar mucosa, which would increase access to host tissue. However, the ability of ChiA-CTD to target and degrade MUC5AC could be beneficial in the context of, for example, asthma where this mucin is overproduced and may underlie the pathology of this disease.

Our finding that ChiA-CTD is capable of mucolytic activity, particularly against MUC5AC, provides a new therapeutic application of chitinase in mucus-driven diseases, such as asthma.

### REFERENCES

1) Hansson, G. C. (2019) Mucus and mucins in diseases of the intestinal and respiratory tracts. Journal of Internal Medicine 285, 479-490.

2) Fahy, J. V. & Dickey, B. F. (2010) Airway mucus function and dysfunction. New. Engl. J. Med. 363, 2233-47.

3) Kuyper, L. M., Pare, P. D., Hogg, J. C., Lambert, R. K., Ionescu, D., Woods, R. & Bai, T. R. (2003) Characterization of airway plugging in fatal asthma. Am. J. Med. 115, 6-11.

4) Bonser, L. R. & Erle, D. J. (2017) Airway Mucus and Asthma: The Role of MUC5AC and MUC5B. J. Clin. Med. 6, pii: E112.

5) Evans, C. M., Raclawska, D. S., Ttofali, F., Liptzin, D. R., Fletcher, A. A., Harper, D. N., McGing, M. A., McElwee, M. M., Williams, O. W., Sanchez, E., Roy, M. G., Kindrachuk, K. N., Wynn, T. A., Eltzschig, H. K., Blackburn, M. R., Tuvim, M. J., Janssen, W. J., Schwartz, D. A. & Dickey, B. F. (2015) The polymeric mucin Muc5ac is required for allergic airway hyperreactivity. Nature Communications 6, 6281.

6) Fanta, C. H. Asthma. (2009) New. Engl. J. Med. 360, 1002-14.

7) DebRoy, S., Dao, J., Soderberg, M., Rossier, O. & Cianciotto, N. P. (2006) Legionella pneumophila type II secretome reveals unique exoproteins and a chitinase that promotes bacterial persistence in the lung. Proc. Natl. Acad. Sci. U.S.A. 103, 19146-51.

8) Frederiksen R F, Paspaliari D K, Larsen T, Storgaard B G, Larsen M H, Ingmer H, et al. Bacterial chitinases and chitin-binding proteins as virulence factors. Microbiology. 2013; 159(Pt 5):833-47. doi: 10.1099/mic.0.051839-0.

9) Luo Q, Kumar P, Vickers T J, Sheikh A, Lewis W G, Rasko D A, et al. Enterotoxigenic Escherichia coli secretes a highly conserved mucin-degrading metalloprotease to effectively engage intestinal epithelial cells. Infect Immun. 2014; 82(2):509-21. doi: 10.1128/IAI.01106-13

10) Baldus S E, Engelmann K, Hanisch F G. MUC1 and the MUCs: a family of human mucins with impact in cancer biology. Crit Rev Clin Lab Sci. 2004; 41(2):189-231. doi: 10.1080/10408360490452040.

11) Evert C, Loesekann T, Bhat G, Shajahan A, Sonon R, Azadi P, et al. Generation of

12) C-Labeled MUC5AC Mucin Oligosaccharides for Stable Isotope Probing of Host-Associated Microbial Communities. ACS Infect Dis. 2019;5(3):385-93. doi: 10.1021/acsinfecdis.8b00296.

13) Noach I, Ficko-Blean E, Pluvinage B, Stuart C, Jenkins M L, Brochu D, et al. Recognition of protein-linked glycans as a determinant of peptidase activity. Proc Natl Acad Sci USA. 2017; 114(5):E679-E88.

14) Hsieh Y C, Wu Y J, Chiang T Y, Kuo C Y, Shrestha K L, Chao C F, et al. Crystal structures of Bacillus cereus NCTU2 chitinase complexes with chitooligomers reveal novel substrate binding for catalysis: a chitinase without chitin binding and insertion domains. J Biol Chem. 2010; 285(41):31603-15.

15) Yu A C, Worrall L J, Strynadka N C. Structural insight into the bacterial mucinase StcE essential to adhesion and immune evasion during enterohemorrhagic E. coli infection. Structure. 2012; 20(4):707-17.

(ChiA-CTD crystal structure amino acid sequence)
SEQ ID NO: 1
GRIIGYVPGWKTPPAAQELASAGYTHVMIAFGVFSTNTPGVIVPAFET
ITKEYIQSLHQAGIKVILSLGGALTSIPNTTVDFHQVLVASSSPEAFK
QTFINSLKELISQYGFDGFDTDIEHGINASGSFSQPQGDIAVLASIIN
TMYSQNSSLLITLTPQVANIAATSGFDQTWGNYASLIMQTHQSLAWVG
IQLYNTGCAFGIDQVCYGPTPTDTPDFSVAMATDLLENWPATVNGRPT
GFQPYISYLRPSQIVIGYPSPNASGGSDGSPVTPTTTIKRAIQCLKTA
IAGNTSCGVYVPPRAYGNIGGVFNWEVTYDKNNQFKFAKELKNCAING
VCE
(ChiA amino acid sequence)
SEQ ID NO: 2
NTSLKPSIAQPTACVNSLFTATGNQHWKSIVLKLTNNCNQAVDFQNST
VSFQTTAALNTSFWGDFSPLSYPDNALNISSQPQSGGNYLATLNLHFP
SYPGANSKLPVGSSISIKYGAVTDSHIEGTANVYLSTTVESGSIQLIN
AAAKPTNVSQGYALVHVTMNGQSVSDVQLPWETSKTLSGFAAGNYAIS
AETVTDSNGNLYLGQANPSMVNVIANQTTSSTINYARVQETGKIKIHV
QNLPGELSNYNENPTVLITQSQSGNSRSQHAVWGNTTTVAELKEGSSY
QFSTPAIQYNDYNCWPTFTPSSLVASAANVPTTNLTYQCAQIVKDNVT
INVSGAPSSLASLKIILTPNDGSQTVEQTIDLANGVGSSAISLTDGVI
YTLSSEGVPGYTVQFSPQPLTATENATVNITLSPVTAAKGRIIGYVPG
WKTPPAAQELASAGYTHVMIAFGVFSTNTPGVIVPAFETITKEYIQSL
HQAGIKVILSLGGALTSIPNTTVDFHQVLVASSSPEAFKQTFINSLKE
LISQYGFDGFDTDIEHGINASGSFSQPQGDIAVLASIINTMYSQNSSL
LITLTPQVANIAATSGFDQTWGNYASLIMQTHQSLAWVGIQLYNTGCA
FGIDQVCYGPTPTDTPDFSVAMATDLLENWPATVNGRPTGFQPYISYL
RPSQIVIGYPSPNASGGSDGSPVTPTTTIKRAIQCLKTAIAGNTSCGV
YVPPRAYGNIGGVFNWEVTYDKNNQFKFAKELKNCAINGVCE
(ChiA-CTD crystal structure nucleic acid
sequence)
SEQ ID NO: 3
GGGCGTATTATTGGTTACGTACCGGGATGGAAAACTCCACCTGCCGCT
CAAGAGTTGGCTAGCGCGGGTTATACTCATGTCATGATTGCTTTCGGC
GTATTTAGTACCAATACTCCGGGTGTTATTGTTCCTGCTTTTGAAACA
ATAACCAAAGAATATATTCAGTCTCTTCATCAAGCCGGGATTAAAGTT
ATTCTTTCTTTAGGTGGTGCGTTAACTAGTATTCCCAATACAACAGTA
GATTTTCACCAGGTTTTAGTAGCTTCTTCTTCACCAGAGGCATTTAAA
CAAACATTTATCAACTCTTTAAAGGAGTTAATTTCTCAATATGGTTTT
GATGGGTTTGATACAGATATTGAGCATGGTATTAACGCTAGCGGTTCC
TTTTCTCAACCACAGGGTGACATTGCTGTCTTAGCAAGCATTATCAAT
ACGATGTACAGCCAAAATTCTTCTCTGCTAATTACTCTGACACCTCAA
GTGGCTAATATTGCTGCAACAAGCGGTTTCGACCAAACCTGGGGGAAT
TATGCCTCTTTAATTATGCAAACCCATCAGTCTTTAGCGTGGGTAGGT
ATCCAGCTTTACAATACAGGATGTGCTTTCGGAATTGATCAAGTATGC
TATGGTCCTACACCAACTGATACCCCTGATTTTTCAGTAGCTATGGCT
ACCGATTTATTGGAGAATTGGCCAGCAACGGTCAATGGACGTCCTACA
GGATTTCAACCTTATATTAGCTATTTAAGACCTTCCCAAATTGTCATT
GGTTATCCATCTCCAAATGCTAGTGGTGGCAGTGACGGTTCACCGGTT
ACTCCGACAACCACAATCAAGCGGGCTATTCAGTGCCTTAAGACAGCA
ATTGCCGGTAATACCAGTTGTGGTGTTTATGTTCCGCCAAGAGCTTAT
GGGAATATCGGTGGTGTATTTAACTGGGAAGTAACTTATGATAAGAAC
AATCAATTCAAATTTGCAAAAGAATTGAAAAATTGTGCTATTAATGGT
GTTTGTGAGTAA
(ChiA nucleic acid sequence)
SEQ ID NO: 4
AATACCTCTTTAAAACCCTCAATTGCCCAGCCTACAGCCTGTGTAAAC
TCACTTTTTACAGCAACGGGAAATCAACACTGGAAATCAATCGTATTA
AAGTTAACCAATAATTGCAATCAGGCAGTCGATTTTCAAAATTCAACT
GTCTCCTTTCAAACAACAGCAGCTTTAAATACCTCTTTTTGGGGGGAT
TTTTCTCCTTTATCTTACCCGGATAATGCTTTAAATATTTCTTCTCAA
CCTCAATCTGGCGGAAACTATCTAGCCACTTTAAACCTGCATTTTCCC
AGCTATCCGGGTGCTAATAGCAAGTTACCTGTCGGAAGTTCTATTTCA
ATTAAATATGGGGCAGTCACTGATAGTCATATCGAGGGAACGGCTAAT
GTTTATTTAAGCACAACGGTTGAATCAGGAAGCATCCAATTAATCAAT
GCTGCTGCAAAACCAACAAATGTGTCACAAGGATATGCATTAGTTCAT
GTCACAATGAATGGTCAATCAGTCAGTGATGTGCAATTACCATGGGAA
ACTTCAAAGACTCTATCTGGGTTTGCTGCAGGTAATTATGCTATTTCT
GCTGAAACGGTTACTGACAGTAATGGCAATCTTTATCTGGGACAGGCC
AATCCCAGCATGGTGAATGTGATTGCAAACCAAACAACGAGCTCGACT
ATTAATTATGCTCGGGTACAAGAGACAGGCAAGATCAAGATCCATGTG
CAGAACCTCCCTGGCGAATTAAGCAACTACAATGAAAATCCAACTGTT
CTAATTACCCAGAGTCAATCAGGAAATTCACGCTCACAGCATGCCGTT
TGGGGGAACACAACGACTGTTGCTGAGTTGAAAGAGGGGAGTAGTTAC
CAATTCTCAACTCCTGCAATTCAATATAACGATTACAATTGCTGGCCC
ACTTTCACACCATCTTCCTTAGTGGCAAGTGCTGCAAATGTTCCAACT
ACCAATTTAACCTATCAGTGTGCCCAGATTGTGAAAGATAATGTGACA
ATTAATGTCAGTGGCGCTCCATCCTCCCTGGCTTCATTGAAAATTATT
TTAACTCCCAACGATGGTTCACAAACAGTAGAACAGACAATAGATTTG
GCCAATGGTGTTGGTTCATCTGCCATTTCTTTAACTGACGGGGTTATT
TATACACTTTCAAGTGAAGGTGTTCCCGGCTATACAGTACAATTTTCG
CCCCAACCTTTAACAGCAACAGAAAATGCAACAGTAAACATCACTTTA
TCTCCGGTAACCGCTGCAAAAGGGCGTATTATTGGTTACGTACCGGGA
TGGAAAACTCCACCTGCCGCTCAAGAGTTGGCTAGCGCGGGTTATACT
CATGTCATGATTGCTTTCGGCGTATTTAGTACCAATACTCCGGGTGTT
ATTGTTCCTGCTTTTGAAACAATAACCAAAGAATATATTCAGTCTCTT
CATCAAGCCGGGATTAAAGTTATTCTTTCTTTAGGTGGTGCGTTAACT
AGTATTCCCAATACAACAGTAGATTTTCACCAGGTTTTAGTAGCTTCT
TCTTCACCAGAGGCATTTAAACAAACATTTATCAACTCTTTAAAGGAG
TTAATTTCTCAATATGGTTTTGATGGGTTTGATACAGATATTGAGCAT
GGTATTAACGCTAGCGGTTCCTTTTCTCAACCACAGGGTGACATTGCT
GTCTTAGCAAGCATTATCAATACGATGTACAGCCAAAATTCTTCTCTG
CTAATTACTCTGACACCTCAAGTGGCTAATATTGCTGCAACAAGCGGT
TTCGACCAAACCTGGGGGAATTATGCCTCTTTAATTATGCAAACCCAT
CAGTCTTTAGCGTGGGTAGGTATCCAGCTTTACAATACAGGATGTGCT
TTCGGAATTGATCAAGTATGCTATGGTCCTACACCAACTGATACCCCT
GATTTTTCAGTAGCTATGGCTACCGATTTATTGGAGAATTGGCCAGCA
ACGGTCAATGGACGTCCTACAGGATTTCAACCTTATATTAGCTATTTA
AGACCTTCCCAAATTGTCATTGGTTATCCATCTCCAAATGCTAGTGGT
GGCAGTGACGGTTCACCGGTTACTCCGACAACCACAATCAAGCGGGCT
ATTCAGTGCCTTAAGACAGCAATTGCCGGTAATACCAGTTGTGGTGTT
TATGTTCCGCCAAGAGCTTATGGGAATATCGGTGGTGTATTTAACTGG
GAAGTAACTTATGATAAGAACAATCAATTCAAATTTGCAAAAGAATTG
AAAAATTGTGCTATTAATGGTGTTTGTGAGTAA

1. A peptide comprising an amino acid sequence having at least 60% sequence identity to the amino acid sequence defined in SEQ ID NO: 1, wherein the peptide has mucolytic activity, wherein the peptide does not consist of the amino sequence defined in SEQ ID NO: 2.

2. The peptide according to claim 1, wherein amino acids at positions equivalent to positions 81, 83, 121, 124, 160, 172, and 194 of SEQ ID NO: 1 are identical to or similar to positions 81, 83, 121, 124, 160, 172, and 194 of SEQ ID NO: 1.

3. The peptide according to claim 1, wherein the peptide consists of an amino acid sequence of no more than 1000 amino acids.

4. The peptide according to claim 1, wherein the peptide retains said mucolytic activity for at least four weeks at 25° C.

5. The peptide according to claim 1, wherein the peptide degrades MUC5AC.

6. The peptide according to claim 1, wherein the peptide is conjugated to at least one other moiety.

7. The peptide according to claim 1, wherein the peptide comprises an amino acid sequence having at least 70%, 75%, 80%, 85%, 90%, 95% or 99% sequence identity to the amino acid sequence defined in SEQ ID NO: 1.

8. A nucleic acid comprising a nucleotide sequence which encodes the peptide according to claim 1.

9. The nucleic acid according to claim 8, wherein the nucleic acid comprises an nucleic acid sequence having at least 60% sequence identity to the nucleic acid sequence defined in SEQ ID NO: 3, wherein the nucleic acid does not consist of the nucleic acid sequence defined in SEQ ID NO: 4.

10. The nucleic acid according to claim 8, wherein the nucleic acid consists of an nucleic acid sequence having at least 70%, 75%, 80%, 85%, 90%, 95% or 99% sequence identity to the nucleic acid sequence defined in SEQ ID NO: 3.

11. A vector comprising a nucleic acid sequence according to claim 8.

12. A host cell comprising a vector according to claim 11 preferably wherein the host cell is E. coli.

13. A method of performing an in vitro assay, the method comprising using a peptide comprising an amino acid sequence having at least 60% sequence identity to the amino acid sequence defined in SEQ ID NO: 1, wherein the peptide has mucolytic activity, or a nucleic acid encoding a peptide having at least 60% sequence identity to the amino acid sequence defined in SEQ ID NO: 1, wherein the peptide has mucolytic activity.

14. A pharmaceutical composition comprising a peptide comprising an amino acid sequence having at least 60% sequence identity to the amino acid sequence defined in SEQ ID NO: 1 wherein the peptide has mucolytic activity, or a nucleic acid encoding a peptide having at least 60% sequence identity to the amino acid sequence defined in SEQ ID NO: 1, wherein the peptide has mucolytic activity, and at least one pharmaceutically acceptable carrier, diluent or excipient.

15. The pharmaceutical composition according to claim 14, wherein the peptide comprises an amino acid sequence having at least 70%, 75%, 80%, 85%, 90%, 95% or 99% sequence identity to the amino acid sequence defined in SEQ ID NO: 1.

16. A method of treatment of a disease or condition characterised by an increased level of mucin, the method comprising the step of administering at least one selected from the group consisting of:
(a) a peptide comprising an amino acid sequence having at least 60% sequence identity to the amino acid sequence defined in SEQ ID NO: 1 wherein the peptide has mucolytic activity,
(b) a nucleic acid encoding a peptide having at least 60% sequence identity to the amino acid sequence defined in SEQ ID NO: 1, wherein the peptide has mucolytic activity, and
(c) a pharmaceutical composition comprising a peptide comprising an amino acid sequence having at least 60% sequence identity to the amino acid sequence defined in SEQ ID NO: 1 wherein the peptide has mucolytic activity, or a nucleic acid encoding a peptide having at least 60% sequence identity to the amino acid sequence defined in SEQ ID NO: 1, wherein the peptide has mucolytic activity,
and at least one pharmaceutically acceptable carrier, diluent or excipient, to a patient in need thereof.

17. The method according to claim 16, wherein amino acids at positions equivalent to positions 81, 83, 121, 124, 160, 172, and 194 of SEQ ID NO: 1 are identical to or similar to positions 81, 83, 121, 124, 160, 172, and 194 of SEQ ID NO: 1.

18. The method according to claim 16, wherein the disease or condition is characterised by an increased level of mucin.

19. The method according to claim 16, wherein the disease or condition is characterised by an increased level of MUC5AC.

20. The method according to claim 16, wherein the disease or condition is a chronic inflammatory lung disease, rhinosinusitis, extramammary Paget's disease, gallstone disease, pancreatic cancer or inflammatory bowel disease.

21. The method according to claim 20 wherein the disease or condition is asthma.


SEQUENCE LISTING (41 sequences)

SEQ ID NO: 1 - protein, 339 aa, Artificial Sequence
  ChiA-CTD crystal structure amino acid sequence
GRIIGYVPGWKTPPAAQELASAGYTHVMIAFGVFSTNTPGVIVPAFETITKEYIQSLHQAGIKVILSLGGALTSIPNTTVDFHQVLVASSSPEAFKQTFINSLKELISQYGFDGFDTDIEHGINASGSFSQPQGDIAVLASIINTMYSQNSSLLITLTPQVANIAATSGFDQTWGNYASLIMQTHQSLAWVGIQLYNTGCAFGIDQVCYGPTPTDTPDFSVAMATDLLENWPATVNGRPTGFQPYISYLRPSQIVIGYPSPNASGGSDGSPVTPTTTIKRAIQCLKTAIAGNTSCGVYVPPRAYGNIGGVFNWEVTYDKNNQFKFAKELKNCAINGVCE

SEQ ID NO: 2 - protein, 762 aa, Legionella pneumophila
NTSLKPSIAQPTACVNSLFTATGNQHWKSIVLKLTNNCNQAVDFQNSTVSFQTTAALNTSFWGDFSPLSYPDNALNISSQPQSGGNYLATLNLHFPSYPGANSKLPVGSSISIKYGAVTDSHIEGTANVYLSTTVESGSIQLINAAAKPTNVSQGYALVHVTMNGQSVSDVQLPWETSKTLSGFAAGNYAISAETVTDSNGNLYLGQANPSMVNVIANQTTSSTINYARVQETGKIKIHVQNLPGELSNYNENPTVLITQSQSGNSRSQHAVWGNTTTVAELKEGSSYQFSTPAIQYNDYNCWPTFTPSSLVASAANVPTTNLTYQCAQIVKDNVTINVSGAPSSLASLKIILTPNDGSQTVEQTIDLANGVGSSAISLTDGVIYTLSSEGVPGYTVQFSPQPLTATENATVNITLSPVTAAKGRIIGYVPGWKTPPAAQELASAGYTHVMIAFGVFSTNTPGVIVPAFETITKEYIQSLHQAGIKVILSLGGALTSIPNTTVDFHQVLVASSSPEAFKQTFINSLKELISQYGFDGFDTDIEHGINASGSFSQPQGDIAVLASIINTMYSQNSSLLITLTPQVANIAATSGFDQTWGNYASLIMQTHQSLAWVGIQLYNTGCAFGIDQVCYGPTPTDTPDFSVAMATDLLENWPATVNGRPTGFQPYISYLRPSQIVIGYPSPNASGGSDGSPVTPTTTIKRAIQCLKTAIAGNTSCGVYVPPRAYGNIGGVFNWEVTYDKNNQFKFAKELKNCAINGVCE

SEQ ID NO: 3 - DNA, 1020 bp, Artificial Sequence
  ChiA-CTD crystal structure nucleic acid sequence
gggcgtattattggttacgtaccgggatggaaaactccacctgccgctcaagagttggctagcgcgggttatactcatgtcatgattgctttcggcgtatttagtaccaatactccgggtgttattgttcctgcttttgaaacaataaccaaagaatatattcagtctcttcatcaagccgggattaaagttattctttctttaggtggtgcgttaactagtattcccaatacaacagtagattttcaccaggttttagtagcttcttcttcaccagaggcatttaaacaaacatttatcaactctttaaaggagttaatttctcaatatggttttgatgggtttgatacagatattgagcatggtattaacgctagcggttccttttctcaaccacagggtgacattgctgtcttagcaagcattatcaatacgatgtacagccaaaattcttctctgctaattactctgacacctcaagtggctaatattgctgcaacaagcggtttcgaccaaacctgggggaattatgcctctttaattatgcaaacccatcagtctttagcgtgggtaggtatccagctttacaatacaggatgtgctttcggaattgatcaagtatgctatggtcctacaccaactgatacccctgatttttcagtagctatggctaccgatttattggagaattggccagcaacggtcaatggacgtcctacaggatttcaaccttatattagctatttaagaccttcccaaattgtcattggttatccatctccaaatgctagtggtggcagtgacggttcaccggttactccgacaaccacaatcaagcgggctattcagtgccttaagacagcaattgccggtaataccagttgtggtgtttatgttccgccaagagcttatgggaatatcggtggtgtatttaactgggaagtaacttatgataagaacaatcaattcaaatttgcaaaagaattgaaaaattgtgctattaatggtgtttgtgagtaa

SEQ ID NO: 4 - DNA, 2289 bp, Legionella pneumophila
aatacctctttaaaaccctcaattgcccagcctacagcctgtgtaaactcactttttacagcaacgggaaatcaacactggaaatcaatcgtattaaagttaaccaataattgcaatcaggcagtcgattttcaaaattcaactgtctcctttcaaacaacagcagctttaaatacctctttttggggggatttttctcctttatcttacccggataatgctttaaatatttcttctcaacctcaatctggcggaaactatctagccactttaaacctgcattttcccagctatccgggtgctaatagcaagttacctgtcggaagttctatttcaattaaatatggggcagtcactgatagtcatatcgagggaacggctaatgtttatttaagcacaacggttgaatcaggaagcatccaattaatcaatgctgctgcaaaaccaacaaatgtgtcacaaggatatgcattagttcatgtcacaatgaatggtcaatcagtcagtgatgtgcaattaccatgggaaacttcaaagactctatctgggtttgctgcaggtaattatgctatttctgctgaaacggttactgacagtaatggcaatctttatctgggacaggccaatcccagcatggtgaatgtgattgcaaaccaaacaacgagctcgactattaattatgctcgggtacaagagacaggcaagatcaagatccatgtgcagaacctccctggcgaattaagcaactacaatgaaaatccaactgttctaattacccagagtcaatcaggaaattcacgctcacagcatgccgtttgggggaacacaacgactgttgctgagttgaaagaggggagtagttaccaattctcaactcctgcaattcaatataacgattacaattgctggcccactttcacaccatcttccttagtggcaagtgctgcaaatgttccaactaccaatttaacctatcagtgtgcccagattgtgaaagataatgtgacaattaatgtcagtggcgctccatcctccctggcttcattgaaaattattttaactcccaacgatggttcacaaacagtagaacagacaatagatttggccaatggtgttggttcatctgccatttctttaactgacggggttatttatacactttcaagtgaaggtgttcccggctatacagtacaattttcgccccaacctttaacagcaacagaaaatgcaacagtaaacatcactttatctccggtaaccgctgcaaaagggcgtattattggttacgtaccgggatggaaaactccacctgccgctcaagagttggctagcgcgggttatactcatgtcatgattgctttcggcgtatttagtaccaatactccgggtgttattgttcctgcttttgaaacaataaccaaagaatatattcagtctcttcatcaagccgggattaaagttattctttctttaggtggtgcgttaactagtattcccaatacaacagtagattttcaccaggttttagtagcttcttcttcaccagaggcatttaaacaaacatttatcaactctttaaaggagttaatttctcaatatggttttgatgggtttgatacagatattgagcatggtattaacgctagcggttccttttctcaaccacagggtgacattgctgtcttagcaagcattatcaatacgatgtacagccaaaattcttctctgctaattactctgacacctcaagtggctaatattgctgcaacaagcggtttcgaccaaacctgggggaattatgcctctttaattatgcaaacccatcagtctttagcgtgggtaggtatccagctttacaatacaggatgtgctttcggaattgatcaagtatgctatggtcctacaccaactgatacccctgatttttcagtagctatggctaccgatttattggagaattggccagcaacggtcaatggacgtcctacaggatttcaaccttatattagctatttaagaccttcccaaattgtcattggttatccatctccaaatgctagtggtggcagtgacggttcaccggttactccgacaaccacaatcaagcgggctattcagtgccttaagacagcaattgccggtaataccagttgtggtgtttatgttccgccaagagcttatgggaatatcggtggtgtatttaactgggaagtaacttatgataagaacaatcaattcaaatttgcaaaagaattgaaaaattgtgctattaatggtgtttgtgagtaa

SEQ ID NO: 5 - protein, 417 aa, Artificial Sequence
  chiA NTD-domain amino acid sequence
NTSLKPSIAQPTACVNSLFTATGNQHWKSIVLKLTNNCNQAVDFQNSTVSFQTTAALNTSFWGDFSPLSYPDNALNISSQPQSGGNYLATLNLHFPSYPGANSKLPVGSSISIKYGAVTDSHIEGTANVYLSTTVESGSIQLINAAAKPTNVSQGYALVHVTMNGQSVSDVQLPWETSKTLSGFAAGNYAISAETVTDSNGNLYLGQANPSMVNVIANQTTSSTINYARVQETGKIKIHVQNLPGELSNYNENPTVLITQSQSGNSRSQHAVWGNTTTVAELKEGSSYQFSTPAIQYNDYNCWPTFTPSSLVASAANVPTTNLTYQCAQIVKDNVTINVSGAPSSLASLKIILTPNDGSQTVEQTIDLANGVGSSAISLTDGVIYTLSSEGVPGYTVQFSPQPLTATENATVNITLS

SEQ ID NO: 6 - DNA, 668 bp, Artificial Sequence
  chiA NTD-domain nucleic acid sequence
cggttactgacagtaatggcaatctttatctgggacaggccaatcccagcatggtgaatgtgattgcaaaccaaacaacgagctcgactattaattatgctcgggtacaagagacaggcaagatcaagatccatgtgcagaacctccctggcgaattaagcaactacaatgaaaatccaactgttctaattacccagagtcaatcaggaaattcacgctcacagcatgccgtttgggggaacacaacgactgttgctgagttgaaagaggggagtagttaccaattctcaactcctgcaattcaatataacgattacaattgctggcccactttcacaccatcttccttagtggcaagtgctgcaaatgttccaactaccaatttaacctatcagtgtgcccagattgtgaaagataatgtgacaattaatgtcagtggcgctccatcctccctggcttcattgaaaattattttaactcccaacgatggttcacaaacagtagaacagacaatagatttggccaatggtgttggttcatctgccatttctttaactgacggggttatttatacactttcaagtgaaggtgttcccggctatacagtacaattttcgccccaacctttaacagcaacagaaaatgcaacagtaaacatcactttatct

SEQ ID NO: 7 - protein, 140 aa, Artificial Sequence
  chiA N1-domain amino acid sequence
NTSLKPSIAQPTACVNSLFTATGNQHWKSIVLKLTNNCNQAVDFQNSTVSFQTTAALNTSFWGDFSPLSYPDNALNISSQPQSGGNYLATLNLHFPSYPGANSKLPVGSSISIKYGAVTDSHIEGTANVYLSTTVESGSI

SEQ ID NO: 8 - DNA, 420 bp, Artificial Sequence
  chiA N1-domain nucleic acid sequence
aatacctctttaaaaccctcaattgcccagcctacagcctgtgtaaactcactttttacagcaacgggaaatcaacactggaaatcaatcgtattaaagttaaccaataattgcaatcaggcagtcgattttcaaaattcaactgtctcctttcaaacaacagcagctttaaatacctctttttggggggatttttctcctttatcttacccggataatgctttaaatatttcttctcaacctcaatctggcggaaactatctagccactttaaacctgcattttcccagctatccgggtgctaatagcaagttacctgtcggaagttctatttcaattaaatatggggcagtcactgatagtcatatcgagggaacggctaatgtttatttaagcacaacggttgaatcaggaagcatc

SEQ ID NO: 9 - protein, 144 aa, Artificial Sequence
  chiA N2-domain amino acid sequence
QLINAAAKPTNVSQGYALVHVTMNGQSVSDVQLPWETSKTLSGFAAGNYAISAETVTDSNGNLYLGQANPSMVNVIANQTTSSTINYARVQETGKIKIHVQNLPGELSNYNENPTVLITQSQSGNSRSQHAVWGNTTTVAELKE

SEQ ID NO: 10 - DNA, 432 bp, Artificial Sequence
  chiA N2-domain nucleic acid sequence
caattaatcaatgctgctgcaaaaccaacaaatgtgtcacaaggatatgcattagttcatgtcacaatgaatggtcaatcagtcagtgatgtgcaattaccatgggaaacttcaaagactctatctgggtttgctgcaggtaattatgctatttctgctgaaacggttactgacagtaatggcaatctttatctgggacaggccaatcccagcatggtgaatgtgattgcaaaccaaacaacgagctcgactattaattatgctcgggtacaagagacaggcaagatcaagatccatgtgcagaacctccctggcgaattaagcaactacaatgaaaatccaactgttctaattacccagagtcaatcaggaaattcacgctcacagcatgccgtttgggggaacacaacgactgttgctgagttgaaagag

SEQ ID NO: 11 - protein, 133 aa, Artificial Sequence
  chiA N3-domain amino acid sequence
GSSYQFSTPAIQYNDYNCWPTFTPSSLVASAANVPTTNLTYQCAQIVKDNVTINVSGAPSSLASLKIILTPNDGSQTVEQTIDLANGVGSSAISLTDGVIYTLSSEGVPGYTVQFSPQPLTATENATVNITLS

SEQ ID NO: 12 - DNA, 399 bp, Artificial Sequence
  chiA N3-domain nucleic acid sequence
gggagtagttaccaattctcaactcctgcaattcaatataacgattacaattgctggcccactttcacaccatcttccttagtggcaagtgctgcaaatgttccaactaccaatttaacctatcagtgtgcccagattgtgaaagataatgtgacaattaatgtcagtggcgctccatcctccctggcttcattgaaaattattttaactcccaacgatggttcacaaacagtagaacagacaatagatttggccaatggtgttggttcatctgccatttctttaactgacggggttatttatacactttcaagtgaaggtgttcccggctatacagtacaattttcgccccaacctttaacagcaacagaaaatgcaacagtaaacatcactttatct

SEQ ID NO: 13 - protein, 344 aa, Artificial Sequence
  ChiA-CTD D504A mutant amino acid sequence
VTAAKGRIIGYVPGWKTPPAAQELASAGYTHVMIAFGVFSTNTPGVIVPAFETITKEYIQSLHQAGIKVILSLGGALTSIPNTTVAFHQVLVASSSPEAFKQTFINSLKELISQYGFDGFDTDIEHGINASGSFSQPQGDIAVLASIINTMYSQNSSLLITLTPQVANIAATSGFDQTWGNYASLIMQTHQSLAWVGIQLYNTGCAFGIDQVCYGPTPTDTPDFSVAMATDLLENWPATVNGRPTGFQPYISYLRPSQIVIGYPSPNASGGSDGSPVTPTTTIKRAIQCLKTAIAGNTSCGVYVPPRAYGNIGGVFNWEVTYDKNNQFKFAKELKNCAINGVCE

SEQ ID NO: 14 - DNA, 1032 bp, Artificial Sequence
  ChiA-CTD D504A mutant nucleic acid sequence
gttaccgcagcaaaaggtcgcattattggttatgttccaggttggaaaacccctccggcagcacaagaactggcaagcgcaggttatacccatgttatgattgcatttggtgtgttcagcaccaatacaccgggtgttattgttccggcatttgaaaccattaccaaagagtatattcagagcctgcatcaggcaggcattaaagttattctgagcttaggtggtgcactgaccagcattccgaataccaccgttgcgtttcatcaggttctggttgcaagcagcagtccggaagcatttaaacagacctttattaacagcctgaaagaactgattagccagtatggttttgatggctttgataccgatattgagcatggtattaatgccagcggtagctttagccagccgcagggtgatattgcagttctggcaagcattattaacaccatgtatagccagaatagcagcctgctgattaccctgacaccgcaggttgcaaatattgcagcaaccagcggttttgatcagacctggggtaattatgcaagcctgattatgcagacccatcagagtctggcatgggttggtattcagctgtataataccggttgtgcctttggtattgatcaggtttgttatggtccgacaccgaccgatacaccggattttagcgttgcaatggcaaccgatctgctggaaaattggcctgcaaccgttaatggtcgtccgaccggttttcagccgtatattagctatctgcgtccgagccagattgtgattggttatccgagtccgaatgcaagcggtggtagtgatggtagtccggttacaccgaccaccaccattaaacgtgcaattcagtgtctgaaaacagccattgcaggtaataccagctgtggtgtttatgttccgcctcgtgcatatggtaatattggtggtgtgtttaattgggaagtgacctacgataaaaacaaccagttcaaattcgccaaagagctgaaaaattgcgcgattaatggcgtgtgtgaa

SEQ ID NO: 15 - protein, 344 aa, Artificial Sequence
  ChiA-CTD H506A mutant amino acid sequence
VTAAKGRIIGYVPGWKTPPAAQELASAGYTHVMIAFGVFSTNTPGVIVPAFETITKEYIQSLHQAGIKVILSLGGALTSIPNTTVDFAQVLVASSSPEAFKQTFINSLKELISQYGFDGFDTDIEHGINASGSFSQPQGDIAVLASIINTMYSQNSSLLITLTPQVANIAATSGFDQTWGNYASLIMQTHQSLAWVGIQLYNTGCAFGIDQVCYGPTPTDTPDFSVAMATDLLENWPATVNGRPTGFQPYISYLRPSQIVIGYPSPNASGGSDGSPVTPTTTIKRAIQCLKTAIAGNTSCGVYVPPRAYGNIGGVFNWEVTYDKNNQFKFAKELKNCAINGVCE

SEQ ID NO: 16 - DNA, 1032 bp, Artificial Sequence
  ChiA-CTD H506A mutant nucleic acid sequence
gttaccgcagcaaaaggtcgcattattggttatgttccaggttggaaaacccctccggcagcacaagaactggcaagcgcaggttatacccatgttatgattgcatttggtgtgttcagcaccaatacaccgggtgttattgttccggcatttgaaaccattaccaaagagtatattcagagcctgcatcaggcaggcattaaagttattctgagcttaggtggtgcactgaccagcattccgaataccaccgttgattttgcgcaggttctggttgcaagcagcagtccggaagcatttaaacagacctttattaacagcctgaaagaactgattagccagtatggttttgatggctttgataccgatattgagcatggtattaatgccagcggtagctttagccagccgcagggtgatattgcagttctggcaagcattattaacaccatgtatagccagaatagcagcctgctgattaccctgacaccgcaggttgcaaatattgcagcaaccagcggttttgatcagacctggggtaattatgcaagcctgattatgcagacccatcagagtctggcatgggttggtattcagctgtataataccggttgtgcctttggtattgatcaggtttgttatggtccgacaccgaccgatacaccggattttagcgttgcaatggcaaccgatctgctggaaaattggcctgcaaccgttaatggtcgtccgaccggttttcagccgtatattagctatctgcgtccgagccagattgtgattggttatccgagtccgaatgcaagcggtggtagtgatggtagtccggttacaccgaccaccaccattaaacgtgcaattcagtgtctgaaaacagccattgcaggtaataccagctgtggtgtttatgttccgcctcgtgcatatggtaatattggtggtgtgtttaattgggaagtgacctacgataaaaacaaccagttcaaattcgccaaagagctgaaaaattgcgcgattaatggcgtgtgtgaa

SEQ ID NO: 17 - protein, 344 aa, Artificial Sequence
  ChiA-CTD E543M mutant amino acid sequence
VTAAKGRIIGYVPGWKTPPAAQELASAGYTHVMIAFGVFSTNTPGVIVPAFETITKEYIQSLHQAGIKVILSLGGALTSIPNTTVDFHQVLVASSSPEAFKQTFINSLKELISQYGFDGFDTDIMHGINASGSFSQPQGDIAVLASIINTMYSQNSSLLITLTPQVANIAATSGFDQTWGNYASLIMQTHQSLAWVGIQLYNTGCAFGIDQVCYGPTPTDTPDFSVAMATDLLENWPATVNGRPTGFQPYISYLRPSQIVIGYPSPNASGGSDGSPVTPTTTIKRAIQCLKTAIAGNTSCGVYVPPRAYGNIGGVFNWEVTYDKNNQFKFAKELKNCAINGVCE

SEQ ID NO: 18 - DNA, 1032 bp, Artificial Sequence
  ChiA-CTD E543M mutant nucleic acid sequence
gttaccgcagcaaaaggtcgcattattggttatgttccaggttggaaaacccctccggcagcacaagaactggcaagcgcaggttatacccatgttatgattgcatttggtgtgttcagcaccaatacaccgggtgttattgttccggcatttgaaaccattaccaaagagtatattcagagcctgcatcaggcaggcattaaagttattctgagcttaggtggtgcactgaccagcattccgaataccaccgttgattttcatcaggttctggttgcaagcagcagtccggaagcatttaaacagacctttattaacagcctgaaagaactgattagccagtatggttttgatggctttgataccgatattatgcatggtattaatgccagcggtagctttagccagccgcagggtgatattgcagttctggcaagcattattaacaccatgtatagccagaatagcagcctgctgattaccctgacaccgcaggttgcaaatattgcagcaaccagcggttttgatcagacctggggtaattatgcaagcctgattatgcagacccatcagagtctggcatgggttggtattcagctgtataataccggttgtgcctttggtattgatcaggtttgttatggtccgacaccgaccgatacaccggattttagcgttgcaatggcaaccgatctgctggaaaattggcctgcaaccgttaatggtcgtccgaccggttttcagccgtatattagctatctgcgtccgagccagattgtgattggttatccgagtccgaatgcaagcggtggtagtgatggtagtccggttacaccgaccaccaccattaaacgtgcaattcagtgtctgaaaacagccattgcaggtaataccagctgtggtgtttatgttccgcctcgtgcatatggtaatattggtggtgtgtttaattgggaagtgacctacgataaaaacaaccagttcaaattcgccaaagagctgaaaaattgcgcgattaatggcgtgtgtgaa

SEQ ID NO: 19 - protein, 344 aa, Artificial Sequence
  ChiA-CTD H544A mutant amino acid sequence
VTAAKGRIIGYVPGWKTPPAAQELASAGYTHVMIAFGVFSTNTPGVIVPAFETITKEYIQSLHQAGIKVILSLGGALTSIPNTTVDFHQVLVASSSPEAFKQTFINSLKELISQYGFDGFDTDIEAGINASGSFSQPQGDIAVLASIINTMYSQNSSLLITLTPQVANIAATSGFDQTWGNYASLIMQTHQSLAWVGIQLYNTGCAFGIDQVCYGPTPTDTPDFSVAMATDLLENWPATVNGRPTGFQPYISYLRPSQIVIGYPSPNASGGSDGSPVTPTTTIKRAIQCLKTAIAGNTSCGVYVPPRAYGNIGGVFNWEVTYDKNNQFKFAKELKNCAINGVCE

SEQ ID NO: 20 - DNA, 1032 bp, Artificial Sequence
  ChiA-CTD H544A mutant nucleic acid sequence
gttaccgcagcaaaaggtcgcattattggttatgttccaggttggaaaacccctccggcagcacaagaactggcaagcgcaggttatacccatgttatgattgcatttggtgtgttcagcaccaatacaccgggtgttattgttccggcatttgaaaccattaccaaagagtatattcagagcctgcatcaggcaggcattaaagttattctgagcttaggtggtgcactgaccagcattccgaataccaccgttgattttcatcaggttctggttgcaagcagcagtccggaagcatttaaacagacctttattaacagcctgaaagaactgattagccagtatggttttgatggctttgataccgatattgaggcgggtattaatgccagcggtagctttagccagccgcagggtgatattgcagttctggcaagcattattaacaccatgtatagccagaatagcagcctgctgattaccctgacaccgcaggttgcaaatattgcagcaaccagcggttttgatcagacctggggtaattatgcaagcctgattatgcagacccatcagagtctggcatgggttggtattcagctgtataataccggttgtgcctttggtattgatcaggtttgttatggtccgacaccgaccgatacaccggattttagcgttgcaatggcaaccgatctgctggaaaattggcctgcaaccgttaatggtcgtccgaccggttttcagccgtatattagctatctgcgtccgagccagattgtgattggttatccgagtccgaatgcaagcggtggtagtgatggtagtccggttacaccgaccaccaccattaaacgtgcaattcagtgtctgaaaacagccattgcaggtaataccagctgtggtgtttatgttccgcctcgtgcatatggtaatattggtggtgtgtttaattgggaagtgacctacgataaaaacaaccagttcaaattcgccaaagagctgaaaaattgcgcgattaatggcgtgtgtgaa

SEQ ID NO: 21 - protein, 344 aa, Artificial Sequence
  ChiA-CTD N547A mutant amino acid sequence
VTAAKGRIIGYVPGWKTPPAAQELASAGYTHVMIAFGVFSTNTPGVIVPAFETITKEYIQSLHQAGIKVILSLGGALTSIPNTTVDFHQVLVASSSPEAFKQTFINSLKELISQYGFDGFDTDIEHGIAASGSFSQPQGDIAVLASIINTMYSQNSSLLITLTPQVANIAATSGFDQTWGNYASLIMQTHQSLAWVGIQLYNTGCAFGIDQVCYGPTPTDTPDFSVAMATDLLENWPATVNGRPTGFQPYISYLRPSQIVIGYPSPNASGGSDGSPVTPTTTIKRAIQCLKTAIAGNTSCGVYVPPRAYGNIGGVFNWEVTYDKNNQFKFAKELKNCAINGVCE

SEQ ID NO: 22 - DNA, 1032 bp, Artificial Sequence
  ChiA-CTD N547A mutant nucleic acid sequence
gttaccgcagcaaaaggtcgcattattggttatgttccaggttggaaaacccctccggcagcacaagaactggcaagcgcaggttatacccatgttatgattgcatttggtgtgttcagcaccaatacaccgggtgttattgttccggcatttgaaaccattaccaaagagtatattcagagcctgcatcaggcaggcattaaagttattctgagcttaggtggtgcactgaccagcattccgaataccaccgttgattttcatcaggttctggttgcaagcagcagtccggaagcatttaaacagacctttattaacagcctgaaagaactgattagccagtatggttttgatggctttgataccgatattgagcatggtattgcggccagcggtagctttagccagccgcagggtgatattgcagttctggcaagcattattaacaccatgtatagccagaatagcagcctgctgattaccctgacaccgcaggttgcaaatattgcagcaaccagcggttttgatcagacctggggtaattatgcaagcctgattatgcagacccatcagagtctggcatgggttggtattcagctgtataataccggttgtgcctttggtattgatcaggtttgttatggtccgacaccgaccgatacaccggattttagcgttgcaatggcaaccgatctgctggaaaattggcctgcaaccgttaatggtcgtccgaccggttttcagccgtatattagctatctgcgtccgagccagattgtgattggttatccgagtccgaatgcaagcggtggtagtgatggtagtccggttacaccgaccaccaccattaaacgtgcaattcagtgtctgaaaacagccattgcaggtaataccagctgtggtgtttatgttccgcctcgtgcatatggtaatattggtggtgtgtttaattgggaagtgacctacgataaaaacaaccagttcaaattcgccaaagagctgaaaaattgcgcgattaatggcgtgtgtgaa

SEQ ID NO: 23 - protein, 344 aa, Artificial Sequence
  ChiA-CTD Q595A mutant amino acid sequence
VTAAKGRIIGYVPGWKTPPAAQELASAGYTHVMIAFGVFSTNTPGVIVPAFETITKEYIQSLHQAGIKVILSLGGALTSIPNTTVDFHQVLVASSSPEAFKQTFINSLKELISQYGFDGFDTDIEHGINASGSFSQPQGDIAVLASIINTMYSQNSSLLITLTPQVANIAATSGFDATWGNYASLIMQTHQSLAWVGIQLYNTGCAFGIDQVCYGPTPTDTPDFSVAMATDLLENWPATVNGRPTGFQPYISYLRPSQIVIGYPSPNASGGSDGSPVTPTTTIKRAIQCLKTAIAGNTSCGVYVPPRAYGNIGGVFNWEVTYDKNNQFKFAKELKNCAINGVCE

SEQ ID NO: 24 - DNA, 1032 bp, Artificial Sequence
  ChiA-CTD Q595A mutant nucleic acid sequence
gttaccgcagcaaaaggtcgcattattggttatgttccaggttggaaaacccctccggcagcacaagaactggcaagcgcaggttatacccatgttatgattgcatttggtgtgttcagcaccaatacaccgggtgttattgttccggcatttgaaaccattaccaaagagtatattcagagcctgcatcaggcaggcattaaagttattctgagcttaggtggtgcactgaccagcattccgaataccaccgttgattttcatcaggttctggttgcaagcagcagtccggaagcatttaaacagacctttattaacagcctgaaagaactgattagccagtatggttttgatggctttgataccgatattgagcatggtattaatgccagcggtagctttagccagccgcagggtgatattgcagttctggcaagcattattaacaccatgtatagccagaatagcagcctgctgattaccctgacaccgcaggttgcaaatattgcagcaaccagcggttttgatgcgacctggggtaattatgcaagcctgattatgcagacccatcagagtctggcatgggttggtattcagctgtataataccggttgtgcctttggtattgatcaggtttgttatggtccgacaccgaccgatacaccggattttagcgttgcaatggcaaccgatctgctggaaaattggcctgcaaccgttaatggtcgtccgaccggttttcagccgtatattagctatctgcgtccgagccagattgtgattggttatccgagtccgaatgcaagcggtggtagtgatggtagtccggttacaccgaccaccaccattaaacgtgcaattcagtgtctgaaaacagccattgcaggtaataccagctgtggtgtttatgttccgcctcgtgcatatggtaatattggtggtgtgtttaattgggaagtgacctacgataaaaacaaccagttcaaattcgccaaagagctgaaaaattgcgcgattaatggcgtgtgtgaa

SEQ ID NO: 25 - protein, 344 aa, Artificial Sequence
  ChiA-CTD Q583A mutant amino acid sequence
VTAAKGRIIGYVPGWKTPPAAQELASAGYTHVMIAFGVFSTNTPGVIVPAFETITKEYIQSLHQAGIKVILSLGGALTSIPNTTVDFHQVLVASSSPEAFKQTFINSLKELISQYGFDGFDTDIEHGINASGSFSQPQGDIAVLASIINTMYSQNSSLLITLTPAVANIAATSGFDQTWGNYASLIMQTHQSLAWVGIQLYNTGCAFGIDQVCYGPTPTDTPDFSVAMATDLLENWPATVNGRPTGFQPYISYLRPSQIVIGYPSPNASGGSDGSPVTPTTTIKRAIQCLKTAIAGNTSCGVYVPPRAYGNIGGVFNWEVTYDKNNQFKFAKELKNCAINGVCE

SEQ ID NO: 26 - DNA, 1032 bp, Artificial Sequence
  ChiA-CTD Q583A mutant nucleic acid sequence
gttaccgcagcaaaaggtcgcattattggttatgttccaggttggaaaacccctccggcagcacaagaactggcaagcgcaggttatacccatgttatgattgcatttggtgtgttcagcaccaatacaccgggtgttattgttccggcatttgaaaccattaccaaagagtatattcagagcctgcatcaggcaggcattaaagttattctgagcttaggtggtgcactgaccagcattccgaataccaccgttgattttcatcaggttctggttgcaagcagcagtccggaagcatttaaacagacctttattaacagcctgaaagaactgattagccagtatggttttgatggctttgataccgatattgagcatggtattaatgccagcggtagctttagccagccgcagggtgatattgcagttctggcaagcattattaacaccatgtatagccagaatagcagcctgctgattaccctgacaccggcggttgcaaatattgcagcaaccagcggttttgatcagacctggggtaattatgcaagcctgattatgcagacccatcagagtctggcatgggttggtattcagctgtataataccggttgtgcctttggtattgatcaggtttgttatggtccgacaccgaccgatacaccggattttagcgttgcaatggcaaccgatctgctggaaaattggcctgcaaccgttaatggtcgtccgaccggttttcagccgtatattagctatctgcgtccgagccagattgtgattggttatccgagtccgaatgcaagcggtggtagtgatggtagtccggttacaccgaccaccaccattaaacgtgcaattcagtgtctgaaaacagccattgcaggtaataccagctgtggtgtttatgttccgcctcgtgcatatggtaatattggtggtgtgtttaattgggaagtgacctacgataaaaacaaccagttcaaattcgccaaagagctgaaaaattgcgcgattaatggcgtgtgtgaa

SEQ ID NO: 27 - protein, 344 aa, Artificial Sequence
  ChiA-CTD Q617A mutant amino acid sequence
VTAAKGRIIGYVPGWKTPPAAQELASAGYTHVMIAFGVFSTNTPGVIVPAFETITKEYIQSLHQAGIKVILSLGGALTSIPNTTVDFHQVLVASSSPEAFKQTFINSLKELISQYGFDGFDTDIEHGINASGSFSQPQGDIAVLASIINTMYSQNSSLLITLTPQVANIAATSGFDQTWGNYASLIMQTHQSLAWVGIALYNTGCAFGIDQVCYGPTPTDTPDFSVAMATDLLENWPATVNGRPTGFQPYISYLRPSQIVIGYPSPNASGGSDGSPVTPTTTIKRAIQCLKTAIAGNTSCGVYVPPRAYGNIGGVFNWEVTYDKNNQFKFAKELKNCAINGVCE

SEQ ID NO: 28 - DNA, 1032 bp, Artificial Sequence
  ChiA-CTD Q617A mutant nucleic acid sequence
gttaccgcagcaaaaggtcgcattattggttatgttccaggttggaaaacccctccggcagcacaagaactggcaagcgcaggttatacccatgttatgattgcatttggtgtgttcagcaccaatacaccgggtgttattgttccggcatttgaaaccattaccaaagagtatattcagagcctgcatcaggcaggcattaaagttattctgagcttaggtggtgcactgaccagcattccgaataccaccgttgattttcatcaggttctggttgcaagcagcagtccggaagcatttaaacagacctttattaacagcctgaaagaactgattagccagtatggttttgatggctttgataccgatattgagcatggtattaatgccagcggtagctttagccagccgcagggtgatattgcagttctggcaagcattattaacaccatgtatagccagaatagcagcctgctgattaccctgacaccgcaggttgcaaatattgcagcaaccagcggttttgatcagacctggggtaattatgcaagcctgattatgcagacccatcagagtctggcatgggttggtattgcgctgtataataccggttgtgcctttggtattgatcaggtttgttatggtccgacaccgaccgatacaccggattttagcgttgcaatggcaaccgatctgctggaaaattggcctgcaaccgttaatggtcgtccgaccggttttcagccgtatattagctatctgcgtccgagccagattgtgattggttatccgagtccgaatgcaagcggtggtagtgatggtagtccggttacaccgaccaccaccattaaacgtgcaattcagtgtctgaaaacagccattgcaggtaataccagctgtggtgtttatgttccgcctcgtgcatatggtaatattggtggtgtgtttaattgggaagtgacctacgataaaaacaaccagttcaaattcgccaaagagctgaaaaattgcgcgattaatggcgtgtgtgaa

SEQ ID NO: 29 - protein, 344 aa, Artificial Sequence
  ChiA-CTD E543M/D504A mutant amino acid sequence
VTAAKGRIIGYVPGWKTPPAAQELASAGYTHVMIAFGVFSTNTPGVIVPAFETITKEYIQSLHQAGIKVILSLGGALTSIPNTTVAFHQVLVASSSPEAFKQTFINSLKELISQYGFDGFDTDIMHGINASGSFSQPQGDIAVLASIINTMYSQNSSLLITLTPQVANIAATSGFDQTWGNYASLIMQTHQSLAWVGIQLYNTGCAFGIDQVCYGPTPTDTPDFSVAMATDLLENWPATVNGRPTGFQPYISYLRPSQIVIGYPSPNASGGSDGSPVTPTTTIKRAIQCLKTAIAGNTSCGVYVPPRAYGNIGGVFNWEVTYDKNNQFKFAKELKNCAINGVCE

SEQ ID NO: 30 - DNA, 1032 bp, Artificial Sequence
  ChiA-CTD E543M/D504A mutant nucleic acid sequence
gttaccgcagcaaaaggtcgcattattggttatgttccaggttggaaaacccctccggcagcacaagaactggcaagcgcaggttatacccatgttatgattgcatttggtgtgttcagcaccaatacaccgggtgttattgttccggcatttgaaaccattaccaaagagtatattcagagcctgcatcaggcaggcattaaagttattctgagcttaggtggtgcactgaccagcattccgaataccaccgttgcgtttcatcaggttctggttgcaagcagcagtccggaagcatttaaacagacctttattaacagcctgaaagaactgattagccagtatggttttgatggctttgataccgatattatgcatggtattaatgccagcggtagctttagccagccgcagggtgatattgcagttctggcaagcattattaacaccatgtatagccagaatagcagcctgctgattaccctgacaccgcaggttgcaaatattgcagcaaccagcggttttgatcagacctggggtaattatgcaagcctgattatgcagacccatcagagtctggcatgggttggtattcagctgtataataccggttgtgcctttggtattgatcaggtttgttatggtccgacaccgaccgatacaccggattttagcgttgcaatggcaaccgatctgctggaaaattggcctgcaaccgttaatggtcgtccgaccggttttcagccgtatattagctatctgcgtccgagccagattgtgattggttatccgagtccgaatgcaagcggtggtagtgatggtagtccggttacaccgaccaccaccattaaacgtgcaattcagtgtctgaaaacagccattgcaggtaataccagctgtggtgtttatgttccgcctcgtgcatatggtaatattggtggtgtgtttaattgggaagtgacctacgataaaaacaaccagttcaaattcgccaaagagctgaaaaattgcgcgattaatggcgtgtgtgaa

SEQ ID NO: 31 - protein, 344 aa, Artificial Sequence
  ChiA-CTD E543M/H506A mutant amino acid sequence
VTAAKGRIIGYVPGWKTPPAAQELASAGYTHVMIAFGVFSTNTPGVIVPAFETITKEYIQSLHQAGIKVILSLGGALTSIPNTTVDFAQVLVASSSPEAFKQTFINSLKELISQYGFDGFDTDIMHGINASGSFSQPQGDIAVLASIINTMYSQNSSLLITLTPQVANIAATSGFDQTWGNYASLIMQTHQSLAWVGIQLYNTGCAFGIDQVCYGPTPTDTPDFSVAMATDLLENWPATVNGRPTGFQPYISYLRPSQIVIGYPSPNASGGSDGSPVTPTTTIKRAIQCLKTAIAGNTSCGVYVPPRAYGNIGGVFNWEVTYDKNNQFKFAKELKNCAINGVCE

SEQ ID NO: 32 - DNA, 1032 bp, Artificial Sequence
  ChiA-CTD E543M/H506A mutant nucleic acid sequence
gttaccgcagcaaaaggtcgcattattggttatgttccaggttggaaaacccctccggcagcacaagaactggcaagcgcaggttatacccatgttatgattgcatttggtgtgttcagcaccaatacaccgggtgttattgttccggcatttgaaaccattaccaaagagtatattcagagcctgcatcaggcaggcattaaagttattctgagcttaggtggtgcactgaccagcattccgaataccaccgttgattttgcgcaggttctggttgcaagcagcagtccggaagcatttaaacagacctttattaacagcctgaaagaactgattagccagtatggttttgatggctttgataccgatattatgcatggtattaatgccagcggtagctttagccagccgcagggtgatattgcagttctggcaagcattattaacaccatgtatagccagaatagcagcctgctgattaccctgacaccgcaggttgcaaatattgcagcaaccagcggttttgatcagacctggggtaattatgcaagcctgattatgcagacccatcagagtctggcatgggttggtattcagctgtataataccggttgtgcctttggtattgatcaggtttgttatggtccgacaccgaccgatacaccggattttagcgttgcaatggcaaccgatctgctggaaaattggcctgcaaccgttaatggtcgtccgaccggttttcagccgtatattagctatctgcgtccgagccagattgtgattggttatccgagtccgaatgcaagcggtggtagtgatggtagtccggttacaccgaccaccaccattaaacgtgcaattcagtgtctgaaaacagccattgcaggtaataccagctgtggtgtttatgttccgcctcgtgcatatggtaatattggtggtgtgtttaattgggaagtgacctacgataaaaacaaccagttcaaattcgccaaagagctgaaaaattgcgcgattaatggcgtgtgtgaa

SEQ ID NO: 33 - protein, 344 aa, Artificial Sequence
  ChiA-CTD E543M/H544A mutant amino acid sequence
VTAAKGRIIGYVPGWKTPPAAQELASAGYTHVMIAFGVFSTNTPGVIVPAFETITKEYIQSLHQAGIKVILSLGGALTSIPNTTVDFHQVLVASSSPEAFKQTFINSLKELISQYGFDGFDTDIMAGINASGSFSQPQGDIAVLASIINTMYSQNSSLLITLTPQVANIAATSGFDQTWGNYASLIMQTHQSLAWVGIQLYNTGCAFGIDQVCYGPTPTDTPDFSVAMATDLLENWPATVNGRPTGFQPYISYLRPSQIVIGYPSPNASGGSDGSPVTPTTTIKRAIQCLKTAIAGNTSCGVYVPPRAYGNIGGVFNWEVTYDKNNQFKFAKELKNCAINGVCE

SEQ ID NO: 34 - DNA, 1032 bp, Artificial Sequence
  ChiA-CTD E543M/H544A mutant nucleic acid sequence
gttaccgcagcaaaaggtcgcattattggttatgttccaggttggaaaacccctccggcagcacaagaactggcaagcgcaggttatacccatgttatgattgcatttggtgtgttcagcaccaatacaccgggtgttattgttccggcatttgaaaccattaccaaagagtatattcagagcctgcatcaggcaggcattaaagttattctgagcttaggtggtgcactgaccagcattccgaataccaccgttgattttcatcaggttctggttgcaagcagcagtccggaagcatttaaacagacctttattaacagcctgaaagaactgattagccagtatggttttgatggctttgataccgatattatggcgggtattaatgccagcggtagctttagccagccgcagggtgatattgcagttctggcaagcattattaacaccatgtatagccagaatagcagcctgctgattaccctgacaccgcaggttgcaaatattgcagcaaccagcggttttgatcagacctggggtaattatgcaagcctgattatgcagacccatcagagtctggcatgggttggtattcagctgtataataccggttgtgcctttggtattgatcaggtttgttatggtccgacaccgaccgatacaccggattttagcgttgcaatggcaaccgatctgctggaaaattggcctgcaaccgttaatggtcgtccgaccggttttcagccgtatattagctatctgcgtccgagccagattgtgattggttatccgagtccgaatgcaagcggtggtagtgatggtagtccggttacaccgaccaccaccattaaacgtgcaattcagtgtctgaaaacagccattgcaggtaataccagctgtggtgtttatgttccgcctcgtgcatatggtaatattggtggtgtgtttaattgggaagtgacctacgataaaaacaaccagttcaaattcgccaaagagctgaaaaattgcgcgattaatggcgtgtgtgaa

SEQ ID NO: 35 - protein, 344 aa, Artificial Sequence
  ChiA-CTD E543M/N547A mutant amino acid sequence
VTAAKGRIIGYVPGWKTPPAAQELASAGYTHVMIAFGVFSTNTPGVIVPAFETITKEYIQSLHQAGIKVILSLGGALTSIPNTTVDFHQVLVASSSPEAFKQTFINSLKELISQYGFDGFDTDIMHGIAASGSFSQPQGDIAVLASIINTMYSQNSSLLITLTPQVANIAATSGFDQTWGNYASLIMQTHQSLAWVGIQLYNTGCAFGIDQVCYGPTPTDTPDFSVAMATDLLENWPATVNGRPTGFQPYISYLRPSQIVIGYPSPNASGGSDGSPVTPTTTIKRAIQCLKTAIAGNTSCGVYVPPRAYGNIGGVFNWEVTYDKNNQFKFAKELKNCAINGVCE

SEQ ID NO: 36 - DNA, 1032 bp, Artificial Sequence
  ChiA-CTD E543M/N547A mutant nucleic acid sequence
gttaccgcagcaaaaggtcgcattattggttatgttccaggttggaaaacccctccggcagcacaagaactggcaagcgcaggttatacccatgttatgattgcatttggtgtgttcagcaccaatacaccgggtgttattgttccggcatttgaaaccattaccaaagagtatattcagagcctgcatcaggcaggcattaaagttattctgagcttaggtggtgcactgaccagcattccgaataccaccgttgattttcatcaggttctggttgcaagcagcagtccggaagcatttaaacagacctttattaacagcctgaaagaactgattagccagtatggttttgatggctttgataccgatattatgcatggtattgcggccagcggtagctttagccagccgcagggtgatattgcagttctggcaagcattattaacaccatgtatagccagaatagcagcctgctgattaccctgacaccgcaggttgcaaatattgcagcaaccagcggttttgatcagacctggggtaattatgcaagcctgattatgcagacccatcagagtctggcatgggttggtattcagctgtataataccggttgtgcctttggtattgatcaggtttgttatggtccgacaccgaccgatacaccggattttagcgttgcaatggcaaccgatctgctggaaaattggcctgcaaccgttaatggtcgtccgaccggttttcagccgtatattagctatctgcgtccgagccagattgtgattggttatccgagtccgaatgcaagcggtggtagtgatggtagtccggttacaccgaccaccaccattaaacgtgcaattcagtgtctgaaaacagccattgcaggtaataccagctgtggtgtttatgttccgcctcgtgcatatggtaatattggtggtgtgtttaattgggaagtgacctacgataaaaacaaccagttcaaattcgccaaagagctgaaaaattgcgcgattaatggcgtgtgtgaa

SEQ ID NO: 37 - protein, 344 aa, Artificial Sequence
  ChiA-CTD E543M/Q595A mutant amino acid sequence
VTAAKGRIIGYVPGWKTPPAAQELASAGYTHVMIAFGVFSTNTPGVIVPAFETITKEYIQSLHQAGIKVILSLGGALTSIPNTTVDFHQVLVASSSPEAFKQTFINSLKELISQYGFDGFDTDIMHGINASGSFSQPQGDIAVLASIINTMYSQNSSLLITLTPQVANIAATSGFDATWGNYASLIMQTHQSLAWVGIQLYNTGCAFGIDQVCYGPTPTDTPDFSVAMATDLLENWPATVNGRPTGFQPYISYLRPSQIVIGYPSPNASGGSDGSPVTPTTTIKRAIQCLKTAIAGNTSCGVYVPPRAYGNIGGVFNWEVTYDKNNQFKFAKELKNCAINGVCE

SEQ ID NO: 38 - DNA, 1032 bp, Artificial Sequence
  ChiA-CTD E543M/Q595A mutant nucleic acid sequence
gttaccgcagcaaaaggtcgcattattggttatgttccaggttggaaaacccctccggcagcacaagaactggcaagcgcaggttatacccatgttatgattgcatttggtgtgttcagcaccaatacaccgggtgttattgttccggcatttgaaaccattaccaaagagtatattcagagcctgcatcaggcaggcattaaagttattctgagcttaggtggtgcactgaccagcattccgaataccaccgttgattttcatcaggttctggttgcaagcagcagtccggaagcatttaaacagacctttattaacagcctgaaagaactgattagccagtatggttttgatggctttgataccgatattatgcatggtattaatgccagcggtagctttagccagccgcagggtgatattgcagttctggcaagcattattaacaccatgtatagccagaatagcagcctgctgattaccctgacaccgcaggttgcaaatattgcagcaaccagcggttttgatgcgacctggggtaattatgcaagcctgattatgcagacccatcagagtctggcatgggttggtattcagctgtataataccggttgtgcctttggtattgatcaggtttgttatggtccgacaccgaccgatacaccggattttagcgttgcaatggcaaccgatctgctggaaaattggcctgcaaccgttaatggtcgtccgaccggttttcagccgtatattagctatctgcgtccgagccagattgtgattggttatccgagtccgaatgcaagcggtggtagtgatggtagtccggttacaccgaccaccaccattaaacgtgcaattcagtgtctgaaaacagccattgcaggtaataccagctgtggtgtttatgttccgcctcgtgcatatggtaatattggtggtgtgtttaattgggaagtgacctacgataaaaacaaccagttcaaattcgccaaagagctgaaaaattgcgcgattaatggcgtgtgtgaa

SEQ ID NO: 39 - protein, 5654 aa, Homo sapiens
MSVGRRKLALLWALALALACTRHTGHAQDGSSESSYKHHPALSPIARGPSGVPLRGATVFPSLRTIPVVRASNPAHNGRVCSTWGSFHYKTFDGDVFRFPGLCNYVFSEHCGAAYEDFNIQLRRSQESAAPTLSRVLMKVDGVVIQLTKGSVLVNGHPVLLPFSQSGVLIQQSSSYTKVEARLGLVLMWNHDDSLLLELDTKYANKTCGLCGDFNGMPVVSELLSHNTKLTPMEFGNLQKMDDPTDQCQDPVPEPPRNCSTGFGICEELLHGQLFSGCVALVDVGSYLEACRQDLCFCEDTDLLSCVCHTLAEYSRQCTHAGGLPQDWRGPDFCPQKCPNNMQYHECRSPCADTCSNQEHSRACEDHCVAGCFCPEGTVLDDIGQTGCVPVSKCACVYNGAAYAPGATYSTDCTNCTCSGGRWSCQEVPCPGTCSVLGGAHFSTFDGKQYTVHGDCSYVLTKPCDSSAFTVLAELRRCGLTDSETCLKSVTLSLDGAQTVVVIKASGEVFLNQIYTQLPISAANVTIFRPSTFFIIAQTSLGLQLNLQLVPTMQLFMQLAPKLRGQTCGLCGNFNSIQADDFRTLSGVVEATAAAFFNTFKTQAACPNIRNSFEDPCSLSVENEKYAQHWCSQLTDADGPFGRCHAAVKPGTYYSNCMFDTCNCERSEDCLCAALSSYVHACAAKGVQLGGWRDGVCTKPMTTCPKSMTYHYHVSTCQPTCRSLSEGDITCSVGFIPVDGCICPKGTFLDDTGKCVQASNCPCYHRGSMIPNGESVHDSGAICTCTHGKLSCIGGQAPAPVCAAPMVFFDCRNATPGDTGAGCQKSCHTLDMTCYSPQCVPGCVCPDGLVADGEGGCITAEDCPCVHNEASYRAGQTIRVGCNTCTCDSRMWRCTDDPCLATCAVYGDGHYLTFDGQSYSFNGDCEYTLVQNHCGGKDSTQDSFRVVTENVPCGTTGTTCSKAIKIFLGGFELKLSHGKVEVIGTDESQEVPYTIRQMGIYLVVDTDIGLVLLWDKKTSIFINLSPEFKGRVCGLCGNFDDIAVNDFATRSRSVVGDVLEFGNSWKLSPSCPDALAPKDPCTANPFRKSWAQKQCSILHGPTFAACHAHVEPARYYEACVNDACACDSGGDCECFCTAVAAYAQACHEVGLCVSWRTPSICPLFCDYYNPEGQCEWHYQPCGVPCLRTCRNPRGDCLRDVRGLEGCYPKCPPEAPIFDEDKMQCVATCPTPPLPPRCHVHGKSYRPGAVVPSDKNCQSCLCTERGVECTYKAEACVCTYNGQRFHPGDVIYHTTDGTGGCISARCGANGTIERRVYPCSPTTPVPPTTFSFSTPPLVVSSTHTPSNGPSSAHTGPPSSAWPTTAGTSPRTRLPTASASLPPVCGEKCLWSPWMDVSRPGRGTDSGDFDTLENLRAHGYRVCESPRSVECRAEDAPGVPLRALGQRVQCSPDVGLTCRNREQASGLCYNYQIRVQCCTPLPCSTSSSPAQTTPPTTSKTTETRASGSSAPSSTPGTVSLSTARTTPAPGTATSVKKTFSTPSPPPVPATSTSSMSTTAPGTSVVSSKPTPTEPSTSSCLQELCTWTEWIDGSYPAPGINGGDFDTFQNLRDEGYTFCESPRSVQCRAESFPNTPLADLGQDVICSHTEGLICLNKNQLPPICYNYEIRIQCCETVNVCRDITRLPKTVATTRPTPHPTGAQTQTTFTTHMPSASTEQPTATSRGGPTATSVTQGTHTTLVTRNCHPRCTWTKWFDVDFPSPGPHGGDKETYNNIIRSGEKICRRPEEITRLQCRAKSHPEVSIEHLGQVVQCSREEGLVCRNQDQQGPFKMCLNYEVRVLCCETPRGCHMTSTPGSTSSSPAQTTPSTTSKTTETQASGSSAPSSTPGTVSLSTARTTPAPGTATSVKKTFSTPSPPPVPATSTSSMSTTAPGTSVVSSKPTPTEPSTSSCLQELCTWTEWIDGSYPAPGINGGDFDTFQNLRDEGYTFCESPRSVQCRAESFPNTPLADLGQDVICSHTEGLICLNKNQLPPICYNYEIRIQCCETVNVCRDITRPPKTVATTRPTPHPTGAQTQTTFTTHMPSASTEQPTATSRGGPTATSVTQGTHTTPVTRNCHPRCTWTTWFDVDFPSPGPHGGDKETYNNIIRSGEKICRRPEEITRLQCRAKSHPEVSIEHLGQVVQCSREEGLVCRNQDQQGPFKMCLNYEVRVLCCETPKGCPVTSTPVTAPSTPSGRATSPTQSTSSWQKSRTTTLVTTSTTSTPQTSTTYAHTTSTTSAPTARTTSAPTTRTTSASPASTTSGPGNTPSPVPTTSTISAPTTSITSAPTTSTTSAPTSSTTSGPGTTPSPVPTTSITSAPTTSTTSAPTTSTTSARTSSTTSATTTSRISGPETTPSPVPTTSTTSATTTSTTSAPTTSTTSAPTSSTTSSPQTSTTSAPTTSTTSGPGTTPSPVPTTSTTSAPTTRTTSAPKSSTTSAATTSTTSGPETTPRPVPTTSTTSSPTTSTTSAPTTSTTSASTTSTTSGAGTTPSPVPTTSTTSAPTTSTTSAPISSTTSATTTSTTSGPGTTPSPVPTTSTTSAPTTSTTSGPGTTPSAVPTTSITSAPTTSTNSAPISSTTSATTTSRISGPETTPSPVPTASTTSASTTSTTSGPGTTPSPVPTTSTISVPTTSTTSASTTSTTSASTTSTTSGPGTTPSPVPTTSTTSAPTTSTTSAPTTSTISAPTTSTTSATTTSTTSAPTPRRTSAPTTSTISASTTSTTSATTTSTTSATTTSTISAPTTSTTLSPTTSTTSTTITSTTSAPISSTTSTPQTSTTSAPTTSTTSGPGTTSSPVPTTSTTSAPTTSTTSAPTTRTTSVPTSSTTSTATTSTTSGPGTTPSPVPTTSTTSAPTTRTTSAPTTSTTSAPTTSTTSAPTSSTTSATTTSTISVPTTSTTSVPGTTPSPVPTTSTISVPTTSTTSASTTSTTSGPGTTPSPVPTTSTTSAPTTSTTSAPTTSTISAPTTSTPSAPTTSTTLAPTTSTTSAPTTSTTSTPTSSTTSSPQTSTTSASTTSITSGPGTTPSPVPTTSTTSAPTTSTTSAATTSTISAPTTSTTSAPTTSTTSASTASKTSGLGTTPSPIPTTSTTSPPTTSTTSASTASKTSGPGTTPSPVPTTSTIFAPRTSTTSASTTSTTPGPGTTPSPVPTTSTASVSKTSTSHVSISKTTHSQPVTRDCHLRCTWTKWFDIDFPSPGPHGGDKETYNNIIRSGEKICRRPEEITRLQCRAESHPEVSIEHLGQVVQCSREEGLVCRNQDQQGPFKMCLNYEVRVLCCETPKGCPVTSTPVTAPSTPSGRATSPTQSTSSWQKSRTTTLVTTSTTSTPQTSTTSAPTTSTTSAPTTSTTSAPTTSTTSTPQTSISSAPTSSTTSAPTSSTISARTTSIISAPTTSTTSSPTTSTTSATTTSTTSAPTSSTTSTPQTSKTSAATSSTTSGSGTTPSPVTTTSTASVSKTSTSHVSVSKTTHSQPVTRDCHPRCTWTKWFDVDFPSPGPHGGDKETYNNIIRSGEKICRRPEEITRLQCRAKSHPEVSIEHLGQVVQCSREEGLVCRNQDQQGPFKMCLNYEVRVLCCETPKGCPVTSTSVTAPSTPSGRATSPTQSTSSWQKSRTTTLVTSSITSTTQTSTTSAPTTSTTPASIPSTTSAPTTSTTSAPTTSTTSAPTTSTTSTPQTTTSSAPTSSTTSAPTTSTISAPTTSTISAPTTSTTSAPTASTTSAPTSTSSAPTTNTTSAPTTSTTSAPITSTISAPTTSTTSTPQTSTISSPTTSTTSTPQTSTTSSPTTSTTSAPTTSTTSAPTTSTTSTPQTSISSAPTSSTTSAPTASTISAPTTSTTSFHTTSTTSPPTSSTSSTPQTSKTSAATSSTTSGSGTTPSPVPTTSTASVSKTSTSHVSVSKTTHSQPVTRDCHPRCTWTKWFDVDFPSPGPHGGDKETYNNIIRSGEKICRRPEEITRLQCRAESHPEVSIEHLGQVVQCSREEGLVCRNQDQQGPFKMCLNYEVRVLCCETPKGCPVTSTPVTAPSTPSGRATSPTQSTSSWQKSRTTTLVTTSTTSTPQTSTTSAPTTSTIPASTPSTTSAPTTSTTSAPTTSTTSAPTHRTTSGPTTSTTLAPTTSTTSAPTTSTNSAPTTSTISASTTSTISAPTTSTISSPTSSTTSTPQTSKTSAATSSTTSGSGTTPSPVPTTSTTSASTTSTTSAPTTSTTSGPGTTPSPVPSTSTTSAATTSTTSAPTTRTTSAPTSSMTSGPGTTPSPVPTTSTTSAPTTSTTSGPGTTPSPVPTTSTTSAPITSTTSGPGSTPSPVPTTSTTSAPTTSTTSASTASTTSGPGTTPSPVPTTSTTSAPTTRTTSASTASTTSGPGSTPSPVPTTSTTSAPTTRTTPASTASTTSGPGTTPSPVPTTSTTSASTTSTISLPTTSTTSAPITSMTSGPGTTPSPVPTTSTTSAPTTSTTSASTASTTSGPGTTPSPVPTTSTTSAPTTSTTSASTASTTSGPGTSLSPVPTTSTTSAPTTSTTSGPGTTPSPVPTTSTTSAPTTSTTSGPGTTPSPVPTTSTTPVSKTSTSHLSVSKTTHSQPVTSDCHPLCAWTKWFDVDFPSPGPHGGDKETYNNIIRSGEKICRRPEEITRLQCRAESHPEVNIEHLGQVVQCSREEGLVCRNQDQQGPFKMCLNYEVRVLCCETPRGCPVTSVTPYGTSPTNALYPSLSTSMVSASVASTSVASSSVASSSVAYSTQTCFCNVADRLYPAGSTIYRHRDLAGHCYYALCSQDCQVVRGVDSDCPSTTLPPAPATSPSISTSEPVTELGCPNAVPPRKKGETWATPNCSEATCEGNNVISLRPRTCPRVEKPTCANGYPAVKVADQDGCCHHYQCQCVCSGWGDPHYITFDGTYYTFLDNCTYVLVQQIVPVYGHFRVLVDNYFCGAEDGLSCPRSIILEYHQDRVVLTRKPVHGVMTNEIIFNNKVVSPGFRKNGIVVSRIGVKMYATIPELGVQVMFSGLIFSVEVPFSKFANNTEGQCGTCTNDRKDECRTPRGTVVASCSEMSGLWNVSIPDQPACHRPHPTPTTVGPTTVGSTTVGPTTVGSTTVGPTTPPAPCLPSPICQLILSKVFEPCHTVIPPLLFYEGCVFDRCHMTDLDVVCSSLELYAALCASHDICIDWRGRTGHMCPFTCPADKVYQPCGPSNPSYCYGNDSASLGALPEAGPITEGCFCPEGMTLFSTSAQVCVPTGCPRCLGPHGEPVKVGHTVGMDCQECTCEAATWTLTCRPKLCPLPPACPLPGFVPVPAAPQAGQCCPQYSCACNTSRCPAPVGCPEGARAIPTYQEGACCPVQNCSWTVCSINGTLYQPGAVVSSSLCETCRCELPGGPPSDAFVVSCETQICNTHCPVGFEYQEQSGQCCGTCVQVACVTNTSKSPAHLFYPGETWSDAGNHCVTHQCEKHQDGLVVVTTKKACPPLSCSLDEARMSKDGCCRFCPPPPPPYQNQSTCAVYHRSLIIQQQGCSSSEPVRLAYCRGNCGDSSSMYSLEGNTVEHRCQCCQELRTSLRNVTLHCTDGSSRAFSYTEVEECGCMGRRCPAPGDTQHSEEAEPEPSQEAESGSWERGVPVSPMH

SEQ ID NO: 40 - protein, 344 aa, Artificial Sequence
  ChiA-CTD amino acid sequence
VTAAKGRIIGYVPGWKTPPAAQELASAGYTHVMIAFGVFSTNTPGVIVPAFETITKEYIQSLHQAGIKVILSLGGALTSIPNTTVDFHQVLVASSSPEAFKQTFINSLKELISQYGFDGFDTDIEHGINASGSFSQPQGDIAVLASIINTMYSQNSSLLITLTPQVANIAATSGFDQTWGNYASLIMQTHQSLAWVGIQLYNTGCAFGIDQVCYGPTPTDTPDFSVAMATDLLENWPATVNGRPTGFQPYISYLRPSQIVIGYPSPNASGGSDGSPVTPTTTIKRAIQCLKTAIAGNTSCGVYVPPRAYGNIGGVFNWEVTYDKNNQFKFAKELKNCAINGVCE

SEQ ID NO: 41 - DNA, 1035 bp, Artificial Sequence
  ChiA-CTD nucleic acid sequence
gtaaccgctgcaaaagggcgtattattggttacgtaccgggatggaaaactccacctgccgctcaagagttggctagcgcgggttatactcatgtcatgattgctttcggcgtatttagtaccaatactccgggtgttattgttcctgcttttgaaacaataaccaaagaatatattcagtctcttcatcaagccgggattaaagttattctttctttaggtggtgcgttaactagtattcccaatacaacagtagattttcaccaggttttagtagcttcttcttcaccagaggcatttaaacaaacatttatcaactctttaaaggagttaatttctcaatatggttttgatgggtttgatacagatattgagcatggtattaacgctagcggttccttttctcaaccacagggtgacattgctgtcttagcaagcattatcaatacgatgtacagccaaaattcttctctgctaattactctgacacctcaagtggctaatattgctgcaacaagcggtttcgaccaaacctgggggaattatgcctctttaattatgcaaacccatcagtctttagcgtgggtaggtatccagctttacaatacaggatgtgctttcggaattgatcaagtatgctatggtcctacaccaactgatacccctgatttttcagtagctatggctaccgatttattggagaattggccagcaacggtcaatggacgtcctacaggatttcaaccttatattagctatttaagaccttcccaaattgtcattggttatccatctccaaatgctagtggtggcagtgacggttcaccggttactccgacaaccacaatcaagcgggctattcagtgccttaagacagcaattgccggtaataccagttgtggtgtttatgttccgccaagagcttatgggaatatcggtggtgtatttaactgggaagtaacttatgataagaacaatcaattcaaatttgcaaaagaattgaaaaattgtgctattaatggtgtttgtgagtaa